SEQUENCE LISTING (19 sequences)

SEQ ID NO: 1 - DNA, 40 bp, Artificial Sequence
  PCR primer
acgtaagcttgccaccatggaaggaggcggcaagcccaac

SEQ ID NO: 2 - DNA, 40 bp, artificial sequence
  DNA primer
acgtaggcggccgctcataaatttgaagcaaatcgtggct

SEQ ID NO: 3 - DNA, 2673 bp, Homo sapiens
atggaaggaggcggcaagcccaactcttcgtctaacagccgggacgatggcaacagcgtcttccccgccaaggcgtccgcgccgggcgcggggccggccgcggccgagaagcgcctgggcaccccgccggggggcggcggggccggcgcgaaggagcacggcaactccgtgtgcttcaaggtggacggcggtggcggcggtggcggcggcggcggcggcggcgaggagccggcggggggcttcgaagacgccgaggggccccggcggcagtacggcttcatgcagaggcagttcacctccatgctgcagcccggggtcaacaaattctccctccgcatgtttgggagccagaaggcggtggaaaaggagcaggaaagggttaaaactgcaggcttctggattatccacccttacagtgatttcaggttttactgggatttaataatgctcataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtttgtggttgacttcatctcatccatcccagtggattatatctttcttattgtagaaaaaggaatggattctgaagtttacaagacagccagggcccttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggattcttcgaggcggcagtatcaagagaagtataagcaagtggaacaatacatgtcattccataagttaccagctgatatgcgtcagaagatacatgattactatgaacacagataccaaggcaaaatctttgatgaggaaaatattctcaatgaactcaatgatcctctgagagaggagatagtcaacttcaactgtcggaaactggtggctacaatgcctttatttgctaatgcggatcctaattttgtgactgccatgctgagcaagttgagatttgaggtgtttcaacctggagattatatcatacgagaaggagccgtgggtaaaaaaatgtatttcattcaacacggtgttgctggtgtcattacaaaatccagtaaagaaatgaagctgacagatggctcttactttggagagatttgcctgctgaccaaaggacgtcgtactgccagtgttcgagctgatacatattgtcgtctttactcactttccgtggacaatttcaacgaggtcctggaggaatatccaatgatgaggagagcctttgagacagttgccattgaccgactagatcgaataggaaagaaaaattcaattcttctgcaaaagttccagaaggatctgaacactggtgttttcaacaatcaggagaacgaaatcctcaagcagattgtgaaacatgacagggagatggtgcaggcaatcgctcccatcaattatcctcaaatgacaaccctgaattccacatcgtctactacgaccccgacctcccgcatgaggacacaatctccaccggtgtacacagcgaccagcctgtctcacagcaacctgcactcccccagtcccagcacacagaccccccagccatcagccatcctgtcaccctgctcctacaccaccgcggtctgcagccctcctgtacagagccctctggccgctcgaactttccactatgcctcccccaccgcctcccagctgtcactcatgcaacagcagccgcagcagcaggtacagcagtcccagccgccgcagactcagccacagcagccgtccccgcagccacagacacctggcagctccacgccgaaaaatgaagtgcacaagagcacgcaggcgcttcacaacaccaacctgacccgggaagtcaggccactctccgcctcgcagccctcgctgccccatgaggtgtccactctgatttccagacctcatcccactgtgggcgagtccctggcctccatccctcaacccgtgacggcggtccccggaacgggccttcaggcagggggcaggagcactgtcccgcagcgcgtcaccctcttccgacagatgtcgtcgggagccatccccccgaaccgaggagtccctccagcaccccctccaccagcagctgctcttccaagagaatcttcctcagtcttaaacacagacccagacgcagaaaagccacgatttgcttcaaatttatga

SEQ ID NO: 4 - protein, 890 aa, Homo sapiens
MEGGGKPNSSSNSRDDGNSVFPAKASAPGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIAPINYPQMTTLNSTSSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPLSASQPSLPHEVSTLISRPHPTVGESLASIPQPVTAVPGTGLQAGGRSTVPQRVTLFRQMSSGAIPPNRGVPPAPPPPAAALPRESSSVLNTDPDAEKPRFASNL

SEQ ID NO: 5 - DNA, 33 bp, Primer
  DNA primer
cctcctccaccacgatgcccgttcggaagtgag

SEQ ID NO: 6 - DNA, 27 bp, Artificial Sequence
  DNA primer
ccatcctaatacgactcactatagggc

SEQ ID NO: 7 - DNA, 41 bp, Artificial Sequence
  DNA primer
atcaaagcttgccaccatggaggcagagcagcggccggcgg

SEQ ID NO: 8 - DNA, 40 bp, Artificial Sequence
  DNA primer
acgtacgcggccgcttacatgttggcagaaagctggagac

SEQ ID NO: 9 - DNA, 2325 bp, Homo sapiens
atggaggcagagcagcggccggcggcgggggccagcgaaggggcgacccctggactggaggcggtgcctcccgttgctcccccgcctgcgaccgcggcctcaggtccgatccccaaatctgggcctgagcctaagaggaggcaccttgggacgctgctccagcctacggtcaacaagttctcccttcgggtgttcggcagccacaaagcagtggaaatcgagcaggagcgggtgaagtcagcgggggcctggatcatccacccctacagcgacttccggttttactgggacctgatcatgctgctgctgatggtggggaacctcatcgtcctgcctgtgggcatcaccttcttcaaggaggagaactccccgccttggatcgtcttcaacgtattgtctgatactttcttcctactggatctggtgctcaacttccgaacgggcatcgtggtggaggagggtgctgagatcctgctggcaccgcgggccatccgcacgcgctacctgcgcacctggttcctggttgacctcatctcttctatccctgtggattacatcttcctagtggtggagctggagccacggttggacgctgaggtctacaaaacggcacgggccctacgcatcgttcgcttcaccaagatcctaagcctgctgaggctgctccgcctctcccgcctcatccgctacatacaccagtgggaggagatctttcacatgacctatgacctggccagtgctgtggttcgcatcttcaacctcattgggatgatgctgctgctatgtcactgggatggctgtctgcagttcctggtgcccatgctgcaggacttccctcccgactgctgggtctccatcaaccacatggtgaaccactcgtggggccgccagtattcccatgccctgttcaaggccatgagccacatgctgtgcattggctatgggcagcaggcacctgtaggcatgcccgacgtctggctcaccatgctcagcatgatcgtaggtgccacatgctacgccatgttcatcggccatgccacggcactcatccagtccctggactcttcccggcgtcagtaccaggagaagtacaagcaggtggagcagtacatgtccttccacaagctgccagcagacacgcggcagcgcatccacgagtactatgagcaccgctaccagggcaagatgttcgatgaggaaagcatcctgggcgagctgagcgagccgcttcgcgaggagatcattaacttcacctgtcggggcctggtggcccacatgccgctgtttgcccatgccgaccccagcttcgtcactgcagttctcaccaagctgcgctttgaggtcttccagccgggggatctcgtggtgcgtgagggctccgtggggaggaagatgtacttcatccagcatgggctgctcagtgtgctggcccgcggcgcccgggacacacgcctcaccgatggatcctactttggggagatctgcctgctaactaggggccggcgcacagccagtgttcgggctgacacctactgccgcctttactcactcagcgtggaccatttcaatgctgtgcttgaggagttccccatgatgcgccgggcctttgagactgtggccatggatcggctgctccgcatcggcaagaagaattccatactgcagcggaagcgctccgagccaagtccaggcagcagtggtggcatcatggagcagcacttggtgcaacatgacagagacatggctcggggtgttcggggtcgggccccgagcacaggagctcagcttagtggaaagccagtactgtgggagccactggtacatgcgccccttcaggcagctgctgtgacctccaatgtggccattgccctgactcatcagcggggccctctgcccctctcccctgactctccagccaccctccttgctcgctctgcttggcgctcagcaggctctccagcttccccgctggtgcccgtccgagctggcccatgggcatccacctcccgcctgcccgccccacctgcccgaaccctgcacgccagcctatcccgggcagggcgctcccaggtctccctgctgggtccccctccaggaggaggtggacggcggctaggacctcggggccgcccactctcagcctcccaaccctctctgcctcagcgggcaacaggcgatggctctcctgggcgtaagggatcaggaagtgagcggctgcctccctcagggctcctggccaaacctccaaggacagcccagccccccaggccaccagtgcctgagccagccacaccccggggtctccagctttctgccaacatgtaa

SEQ ID NO: 10 - protein, 774 aa, Homo sapiens
MEAEQRPAAGASEGATPGLEAVPPVAPPPATAASGPIPKSGPEPKRRHLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPPDCWVSINHMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLLRIGKKNSILQRKRSEPSPGSSGGIMEQHLVQHDRDMARGVRGRAPSTGAQLSGKPVLWEPLVHAPLQAAAVTSNVAIALTHQRGPLPLSPDSPATLLARSAWRSAGSPASPLVPVRAGPWASTSRLPAPPARTLHASLSRAGRSQVSLLGPPPGGGGRRLGPRGRPLSASQPSLPQRATGDGSPGRKGSGSERLPPSGLLAKPPRTAQPPRPPVPEPATPRGLQLSANM

SEQ ID NO: 11 - DNA, 24 bp, Artificial Sequence
  DNA primer
agcttcgtcactgcagttctcacc

SEQ ID NO: 12 - DNA, 25 bp, Artificial Sequence
  DNA primer
agccatgtctctgtcatgttgcacc

SEQ ID NO: 13 - DNA, 21 bp, Artificial Sequence
  DNA primer
agtggcaccttccagggtcaa

SEQ ID NO: 14 - DNA, 30 bp, Artificial Sequence
  DNA primer
gcggatccccggacctcggggccgcccact

SEQ ID NO: 15 - DNA, 30 bp, Artificial Sequence
  DNA primer
gcgaattctcacatgttggcagaaatttgg

SEQ ID NO: 16 - protein, 69 aa, Homo sapiens
GPRGRPLSASQPSLPQRATGDGSPRRKGSGSERLPPSGLLAKPPGTVQPSRSSVPEPVTPRGPQISANM

SEQ ID NO: 17 - DNA, 27 bp, Artificial Sequence
  DNA primer
gcggatccccacagtccacagcactgg

SEQ ID NO: 18 - DNA, 29 bp, Artificial Sequence
  DNA primer
gcgaattctcataaattcgaagcaaaacg

SEQ ID NO: 19 - protein, 69 aa, Homo sapiens
TVHSTGLQAGSRSTVPQRVTLFRQMSSGAIPPNRGVPPAPPPPAAVQRESPSVLNKDPDAEKPRFASNL

(19) United States
(12) Patent Application Publication
(10) Pub. No.: US 2003/0022813 A1
(43) Pub. Date: Jan. 30, 2003

(54) Treating pain by targeting hyperpolarization-activated, cyclic nucleotide-gated channels

(76) Inventors: Sandra Chaplan, San Diego, CA (US); Adrienne Dubin, San Diego, CA (US); Hong-Qing Guo, San Diego, CA (US); Doo Hyun Lee, Cardiff, CA (US); Changlu Liu, San Diego, CA (US); Lin Luo, La Jolla, CA (US); Sean Brown, Encinitas, CA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/158,711

(22) Filed: May 30, 2002

Related U.S. Application Data
(60) Provisional application No. 60/297,108, filed on Jun. 8, 2001.
(60) Provisional application No. 60/347,945, filed on Nov. 7, 2001.
(60) Provisional application No. 60/373,012, filed on Apr. 16, 2002.

(51) Int. Cl. A61K 31/00

(52) U.S. Cl. 514/1

(57) ABSTRACT

Markedly enhanced activity of pacemaker (hyperpolarization-activated, cation-nonselective, HCN) ion channels governs spontaneous firing in sensory cells of allodynic rats. An HCN ion channel specific blocker, ZD7288, dose-dependently and completely suppresses allodynia. Nerve injury increases the population of large DRG neurons expressing a high density of Iₕ and modulates HCN mRNA expression. New methods of treating pain by targeting HCN pacemaker channels are developed. In addition, new methods for identifying compositions useful for treating pain are disclosed.

### FIELD OF THE INVENTION

This Patent application claims priority from U.S. Provisional Patent Application No. 60/297,108 filed Jun. 8, 2001 entitled “DRG 1H and HCN Association with Neuropathic Pain”, from U.S. Provisional Patent Application No. 60/347,945 filed Nov. 7, 2001 and entitled “Markedly Increased 1H and Regulation of HCN accompany Neuropathic Pain in the Rat”, and from U.S. Provisional Patent Application No. 60/373,012 filed Apr. 16, 2002 entitled “Treating Pain By Targeting Hyperpolarization-Activated, Cyclic Nucleotide-Gated Channels”, the contents of which are hereby incorporated herein in their entirety.

The present invention relates to treatment of pain. More particularly, the present invention relates to using hyperpolarization-activated, cyclic nucleotide-gated (HCN pacemaker) channels as therapeutic targets for the treatment of neuropathic pain and inflammatory pain.

### BACKGROUND OF THE INVENTION

Pain can be devastating to the sufferer. The causes of pain can include inflammation, injury, disease, muscle spasm and the onset of a neuropathic event or syndrome. Generally, pain is experienced when bodily tissues are subjected to mechanical, thermal or chemical stimuli of sufficient intensity to be capable of producing tissue damage. Pain resolves when the stimulus is removed or the injured tissue heals. However, under conditions of inflammatory sensitization or damage to actual nerve tissue, spontaneous pain may become chronic or permanent despite apparent tissue healing. Pain may be felt in the absence of an external stimulus and the pain experienced due to stimuli may become disproportionately intense and persistent.

Inflammatory pain can result from surgery, an adverse physical, chemical or thermal event, infection by a biologic agent, and/or idiopathic/autoimmune processes. Causes of inflammatory pain are numerous and include, but are not limited to, infections, burn pain, rheumatoid arthritis, inflammatory arthritis, ankylosing spondylitis, osteoarthritis, colitis, irritable bowel disease, carditis, dermatitis, myositis, neuritis and collagen vascular diseases, as well as cancer. Current methods for treating inflammatory pain have many drawbacks and deficiencies. For example, corticosteroids, which are commonly used to suppress destructive autoimmune processes, can result in undesirable side effects including, but not limited to, vulnerability to infection, weakening of tissues and loss of bone density leading to fractures, and ocular cataract formation.

Neuropathic pain is defined as pain induced by injury or disease of the peripheral or central nervous system. Neuropathic pain conditions are heterogeneous and include, but are not limited to, mechanical nerve injury, e.g., carpal tunnel syndrome, radiculopathy due to intervertebral disk herniation; post-amputation syndromes, e.g. stump pain, phantom limb pain; metabolic disease, e.g., diabetic neuropathy; neurotropic viral disease, e.g., herpes zoster, human immunodeficiency virus (HIV) disease; cancer, e.g. tumor infiltration, irritation or compression of nervous tissue; radiation neuritis, as after cancer radiotherapy; neurotoxicity, e.g., caused by exogenous substances such as chemotherapy of cancer, HIV or tuberculosis; inflammatory and/or immunologic mechanisms, e.g., multiple sclerosis, paraneoplastic syndromes; nervous system focal ischemia, e.g., thalamic syndrome (anesthesia dolorosa); multiple neurotransmitter system dysfunction, e.g., complex regional pain syndrome (CRPS); and idiopathic causes, e.g., trigeminal neuralgia.

The long-term treatment of chronic pain of any etiology may be very challenging. Although pain may respond to conventional analgesics, the side effects may not be tolerable, or tolerance to the analgesic effects of the drug in question may render therapy problematic. Therapy with ibuprofen and aspirin (both nonsteroidal anti-inflammatory drugs) may be limited by gastrointestinal side effects. Chronic therapy with opiate drugs (morphine, codeine, hydrocodone, oxycodone, etc. and derivatives) may be unacceptable to either the patient or the physician due to side effects (sedation, constipation, etc.), the difficulties of pain management associated with drug tolerance or withdrawal phenomena, and to social factors (the stigma of opiate consumption, concerns about substance abuse potential, drug diversion, loss of productivity, etc).

It is well known to both preclinical investigators and clinicians that neuropathic pain is particularly difficult to treat. Commonly used analgesics such as opiates and nonsteroidal anti-inflammatory drugs are often ineffective to alleviate neuropathic pain. For morphine-like drugs (opiates), perceived efficacy may have to do with sedation (i.e., the patient is too sedated to care about pain). Also, the use of opiates to treat neuropathic pain may be more likely to be associated with tolerance and escalating dose requirements that render therapy problematic. Therefore, the analgesic effects of these compounds may be transient. The vast majority of patients treated with these analgesics continue to experience pain and may not experience pain relief at all. A number of so-called “adjuvant” analgesics, drugs not typically thought of as pain relievers, such as the tricyclic antidepressants (e.g. amitriptyline, nortriptyline, desipramine, imipramine), certain anticonvulsants (e.g. carbamazepine, gabapentin, phenytoin, lamotrigine), the antiarrythmic drugs mexiletine, lidocaine, and tocainide, and various miscellaneous drugs such as baclofen (GABA-B agonist) and clonidine (alpha2 adrenergic agonist) have become the mainstays of neuropathic pain therapy. These agents, however, also suffer from limited efficacy or significant side effects ranging from sedation to cardiovascular effects to life-threatening bone marrow suppression. A number of invasive treatments exist, both pharmacological (nerve blocks, spinal injections, implantable drug delivery devices) and non-pharmacological (e.g. implantable nerve/spinal cord stimulators, neuroablative procedures); all suffer from both limited efficacy and the drawback of the known potential for complications due to the respective procedures. Limitations of the current armamentarium of analgesics call for development of novel methods and strategies with original mechanisms for the treatment of neuropathic pain.

Despite the diversity of etiologies, many neuropathic pain syndromes share common clinical characteristics. Symptoms of neuropathic pain include unusual sensations of burning, tingling, electricity, pins and needles, stiffness, numbness in the extremities, feelings of bodily distortion, allodynia (pain evoked by innocuous stimulation of the skin), hyperalgesia (lowered threshold for pain, e.g. mild thermal stimuli cause pain) hyperpathia (an elevated pain threshold however with an exaggerated pain response once the threshold is surpassed) summation (cumulative exacerbation of pain with repetitive mild stimuli), and pain in the absence of other sensory function in the affected area. These observations have led to the proposal that many neuropathic pain syndromes may share common mechanisms.

Experiments using various animal models have suggested that spontaneous activity in the peripheral and/or central nervous system could be a mechanism by which pain can be explained. A consistent observation from animal model studies is that primary afferent neurons in the dorsal root ganglia of affected spinal levels demonstrate spontaneous discharges. These discharges are predominantly associated with Aβ and Aδ fibers although enhanced C fiber activity may also be involved. Additional studies have demonstrated spontaneous or abnormally easily evoked discharges in second-order neurons in the spinal cord dorsal horn upon which these primary afferent neurons synapse. Consequently, it is held that treatments that suppress the spontaneous discharges or abnormal excitability will thereby decrease pain (Gold, (2000) Pain 84: 117-20). In particular, drugs or compounds that selectively suppress spontaneous/hyperexcitable discharges without interfering with other normal neuronal transmission are likely to be useful in the treatment of pain syndromes.

### SUMMARY OF THE INVENTION

This invention teaches the role of a hithertofore unknown cellular component involved in pain: HCN pacemaker channels, which can serve as a specific therapeutic target for developing novel treatment for pain, preferably neuropathic or inflammatory pain.

In one aspect, the present invention relates to a method for preventing the onset of pain in a subject in need thereof, comprising administering to the subject a prophylactically effective dose of a composition that decreases the current mediated by an HCN pacemaker channel, or the expression of an HCN subunit, in a sensory cell of the subject, in the presence or absence of one or more other analgesics.

In another aspect, the present invention relates to a method for treating pain in a subject in need thereof, comprising administering to the subject a therapeutically effective dose of a composition that decreases the current density of a current mediated by an HCN pacemaker channel, or the expression of an HCN subunit, in a sensory cell of the subject, in the presence or absence of one or more other analgesics.

In another aspect, the present invention relates to a method of identifying a compound useful for treating pain, comprising the steps of:

(a) contacting a test compound with an HCN pacemaker protein; and

(b) determining the ability of the compound to decrease HCN-pacemaker channel-mediated currents.

Optionally the method can be further confirmed through the addition of an additional step comprising: administering the compound to an animal model for pain.

The present invention relates to another method of identifying a compound useful for treating pain, comprising the steps of:

(a) contacting a test compound with a regulatory sequence for an HCN pacemaker gene or a protein that binds to the regulatory sequence for an HCN pacemaker gene; and

(b) determining whether the test compound decreases the expression of the HCN gene controlled by said regulatory sequence.

Optionally the method can be further confirmed through the addition of an additional step comprising: administering the compound to an animal model for pain.

The present invention relates to yet another method of identifying a compound useful for treating pain, comprising the steps of:

(a) combining a test compound, a measurably labeled ligand for an HCN pacemaker protein, and an HCN pacemaker protein; and

(b) measuring binding of the compound to the HCN pacemaker protein by a reduction in the amount of labeled ligand binding to the HCN pacemaker protein.

Optionally the method can be further confirmed through the addition of an additional step comprising: administering the compound to an animal model for pain.

Also included in the present invention is an antibody that binds specifically to the carboxy-terminus of a HCN protein.

Other features and advantages of the invention will be apparent from the following detailed description and claims.

### BRIEF DESCRIPTION OF THE DRAWINGS

FIG. 1. Hyperpolarization-activated currents were elicited in Xenopus oocytes previously injected with human HCN1 cRNA (HCN1) or water (control). The figure shows the mean+/−SEM inward current at the indicated test pulse potential at the end of an 800 msec voltage step. Inward currents were elicited in HCN1-injected oocytes by hyperpolarization steps equal to or greater than −60 mV (squares; n=4 oocytes from 2 separate batches of oocytes). There were no detectable time-dependent inward currents until ˜−140 mV in control sister oocytes (triangle; n=8 oocytes from 2 separate batches of oocytes). At voltage steps in the physiological range, there was a significant difference (asterisks indicate p<0.05; Student's t-test). Reproducible results were obtained from two separate oocyte batches and the data were pooled.

FIG. 2. Low threshold hyperpolarization-activated currents were elicited in HEK293 cells stably expressing human HCN3. (a). Slowly activating inward currents were elicited by the voltage protocol shown in (b). The current-voltage relationship (c) reveals a threshold voltage for activation near −84 mV (note that the voltage axis includes the junction potential correction of −14 mV). (d). No inward currents are observed in control cells (same voltage protocol as shown in (b)). Intracellular solution: K gluconate IS; Extracellular solution: Tyrode's. Holding potential was −64 mV.

FIG. 3. The figure illustrates data obtained using an in-continuity preparation of excised dorsal root, dorsal root ganglion and spinal nerve from rats having been prepared with L4/5 spinal nerve ligation (SNL) 1-3 weeks previously. Spontaneous discharges were recorded in vitro in an ACSF bath (see Example 4). In panels (a) and (b), examples of the effect of bath application of 100 micromolar ZD7288 (a specific blocker of Iₕ; (BoSmith et al., (1993) Br J Pharmacol 110: 343-9)) on spontaneous firing of Aβ and Aδ neurons (distinguished by conduction velocity) are illustrated. Panel (a): Histogram (y-axis=spikes/second) for single fiber in vitro recording from a typical Aβ fiber before and after application of ZD7288 100 micromolar shows that complete suppression of ectopic firing was achieved after 3-4 minutes. The horizontal bar above the histogram indicates the timing and duration of application of ZD7288 to the preparation. Inset (a)(i) shows an enlarged view of a one-second recording period prior to drug application, illustrating baseline spike frequency; inset (a)(ii) shows a one-second recording period after ZD7288 application, illustrating the reduction in firing. The conduction velocity for the depicted fiber was 31.3 m/sec (Aβ range) Panel (b): Single fiber recording as in (a) from an Aδ fiber shows attenuation of firing after ZD7288 application. The horizontal bar above the histogram indicates the timing and duration of application of ZD7288 to the preparation. Inset (b)(i) shows an enlarged view of a one-second recording period prior to drug application, illustrating baseline spike frequency; inset (b)(ii) shows a one second recording period after ZD7288 application, illustrating the degree of reduction in firing. The conduction velocity for the depicted fiber was 7.8 m/sec (A∂ range).

FIG. 4. This graph shows the time course (x-axis) of percent change in firing from baseline (y-axis), in the single fiber recording experiments illustrated in FIG. 3 (above), after ZD7288 application. Horizontal bar between 0-5 minutes indicates timing and duration of ZD7288 100 micromolar application. Data points and error bars indicate mean+/−SEM for 7-8 fibers per group. Symbols: Filled squares=ACSF control (Aβ and Aδ fibers combined); open squares=Aδ fibers; open circles=Aβ fibers. *=P<0.05, 1-way ANOVA, followed by Dunnett's multiple comparisons.

FIG. 5. Allodynia exhibited by SNL rats was blocked in a dose dependent manner by i.p. administration of ZD7288, 1 mg/kg (open squares), 3 mg/kg (open circles), or 10 mg/kg (filled squares), compared with saline vehicle (filled circles). (a) Y-axis shows 50% threshold for paw withdrawal from von Frey hairs; X-axis shows a redacted time course illustrating pre-ligation baseline paw threshold (normal), immediate pre-drug baseline threshold (maximum allodynia, “base”) and post-drug administration time points. The timing of drug administration is indicated by the arrow. (b) the same data analyzed as a dose-response curve showing the ED50 of ZD7288 to be ˜3 mg/kg for allodynia suppression. To compare dose and drug effects, raw paw thresholds were normalized as percent of maximum possible drug effect (% MPE, Y-axis) using the following formula: % MPE=[post-drug threshold (g)−predrug allodynia baseline threshold (g)]/[Pre-ligation baseline threshold (g)]−predrug allodynia baseline threshold (g)]×100. Pre-drug maximum allodynia (baseline) thresholds were assumed to reflect 0% drug effect (no suppression of allodynia) and pre-ligation threshold values were designated as 100% effect, i.e., a drug effect causing return of the paw threshold to a normal, pre-ligation baseline was taken to represent complete suppression of allodynia.

FIG. 6. Graph illustrates the minimal effect of i.p. ZD7288 10 mg/kg (filled circles) on acute thermally-evoked pain, as determined using the 55° C. hot plate test, in which the latency to demonstrating escape behavior (hindpaw licking) from a noxious thermal stimulus is timed, in normal rats, with and without drug treatment. Filled circles; ZD7288 10 mg/kg, i.p. (N=8); open circles; saline vehicle i.p. (N=8). *=P<0.05, t-test for 75 min timepoint only; N=8 per group.

FIG. 7. In the rat complete Freund's adjuvant (CFA) paw model of inflammatory pain, allodynia was suppressed by HCN blockade with ZD7288, as well as by treatment with morphine and ibuprofen, but not by gabapentin. All drugs were administered i.p. Symbols: ZD7288 10 mg/kg, filled circles; ibuprofen 30 mg/kg, open circles; morphine 3 mg/kg, filled triangles; gabapentin 100 mg/kg, open triangles. Y-axis: paw withdrawal threshold (g). X-axis: Normal baseline thresholds, maximum allodynia timepoint after CFA administration, and timepoints after drug treatment. N=6 per group.

FIG. 8. Spontaneous pain behaviors were blocked in the rat mild thermal injury model. Drugs (morphine, ZD7288, or saline) were administered 10 minutes after the mild thermal injury. The total amount of time during which spontaneous pain behaviors were displayed (paw lifting, paw shaking, guarding posture of paw) during two separate 10-minute intervals, 30 and 60 minutes after intraperitoneal vehicle or drug administration, was recorded.

Panel a: Raw data are presented. Y-axis: cumulative spontaneous pain behavior time (seconds). X-axis: time post drug administration (hrs). Both morphine (hatched bars, n=3) and ZD7288 (filled bars, n=6) showed near complete suppression of spontaneous pain behaviors compared to saline (open bars, n=9) at both 30 and 60 min after administration (*=P<0.0001, one-way ANOVA).

Panel b: Data were converted to percent efficacy (versus saline). Mean percent efficacy (Y-axis) (0=no effect, 100%=complete suppression of spontaneous pain) was calculated as (1−(observed pain score/mean overall saline pain score))×100; percent efficacy for the two timepoints was averaged. Percent overall efficacy for morphine was 89.6+/−2.1 (mean+/−SEM), for ZD7288 89.1+/−15.7; *=P<0.0001 vs. saline, 1 way ANOVA with Fisher's PLSD.

FIG. 9. Quantitative RT-PCR analysis of HCN mRNAs in the cell bodies of primary afferent neurons of nerve-injured (SNL) and control (Sham) rats. The relative abundance of the four HCN subtypes was simultaneously measured in whole L5/6 DRGs from 1-week nerve ligated versus sham control rats. The Y-axis represents relative mRNA copy number as detected by fluorescence, normalized to the housekeeping gene cyclophilin A. Panel (A): A significant decrease in HCN1 mRNA in SNL samples, compared to sham controls, was seen using primers that amplified a region toward the 3′ end of the coding sequence (labeled 3′ in figure), whereas no significant change in the abundance of a 5′ region amplicon (labeled 5′ in figure) spanning the region of intron #1(Ludwig et al., (1999) Embo J 18: 2323-9) was seen. Panel (B): A significant decrease in HCN2 mRNA was seen in SNL samples for the amplicon in the region of intron #1 (as above). Panel (C) No significant difference between SNL and control was observed for HCN3, again, using an amplicon in the region of intron #1. Panel (D): No significant difference between SNL and control was observed for an amplicon in this region for HCN4. N=8 SNL and 8 sham control rats. Asterisks indicate P<0.02, unpaired t-test.

FIG. 10. Iₕ was detected in both control (hatched bars) and SNL (solid bars) L5 large neurons at a step to −114 mV. The distribution of Iₕ peak current in large DRG neurons is shown, normalized to cell size (current density). A much larger population of neurons expressed high levels of Iₕ in SNL operated rats compared to sham controls.

FIG. 11. The voltage dependence of Iₕ activation was determined using tail current analysis in which the current through channels that were opened by a previous voltage step was measured before they deactivated. Open circles represent sham neurons, and filled circles represent SNL neurons. Tail currents were determined from a step to −64 or −54 mV after >2 sec duration pre-pulses to a series of voltages between −44 and −154 mV in −10 mV increments. The voltages at which tail currents were measured (−64 or −54 mV) were chosen because tail currents were large enough to provide accurate measurements and there was little contamination by other voltage-gated channel currents. The data were normalized to the maximum tail current observed after the most hyperpolarizing prepulses (y axis: 1 is maximum current), then were fit by a Boltzmann function, and the voltage to half maximal activation (V_{0.5}) and slope of the curve were determined. The threshold for activation was estimated from these plots and was similar to the values determined by measuring current at the end of >2 sec test steps where the threshold in neurons of SNL rats was significantly more positive (Mean+/−SEM: −64.3+/−1.0 mV, n=44) compared to controls (73.9+/−1.9 mV, n=35; p<<0.001). V_{0.5} was calculated to be −82.5+/−2.9 mV in SNL neurons (N=17); this was also significantly different from controls, −91.0+/−2.6 (P<0.05, t-test). Slopes, however, did not differ significantly, at 9.5+/−1.1 for SNLs (N=15) and 9.3+/−1.1 for controls (N=11).

FIG. 12. The effect of bath-applied lidocaine-HCl at neutral pH on native Iₕ in normal rat L4 dorsal root ganglion neurons (large neurons, diameter >42 microns) is illustrated. Y-axis: percent inhibition of current at −134 mV. X-axis: concentration of lidocaine-HCl (M) expressed in log. Concentration-dependent block of Iₕ was seen with an ED50 of 23 micromolar. Data were obtained from 3 cells.

### DETAILED DESCRIPTION OF THE INVENTION

The present invention relates to the treatment of pain. Particularly, the present invention provides a new therapeutic target, the HCN pacemaker channel, for developing novel methods and strategies for treatment of pain, preferably neuropathic pain or inflammatory pain. HCN pacemaker channels are involved in pain.

Hyperpolarization-activated, cyclic nucleotide-gated (HCN) channels have recently been identified as a family of pacemaker channels responsible for fast rhythmic oscillations inherent in cardiac and neuronal depolarizations.

The pacemaker current is a hyperpolarization-activated, cation-selective, inward current that modulates the firing rate of cardiac and neuronal pacemaker cells. This current is of ten times abbreviated as Iₕ (“hyperpolarization”), I_{f} (“funny”), or I_{q} (“queer”). Iₕ contributes to normal pacemaking in the sinoatrial node and atrioventricular node of the heart and Purkinje fibers in the ventricle (DiFrancesco, (1995) Acta Cardiol 50: 413-27), and to abnormal automatic activity of cardiac myocytes under pathological conditions (Opthof, (1998) Cardiovasc Res 38: 537-40.). Iₕ also mediates repetitive firing in neurons and oscillatory behavior in neuronal networks. In addition, it acts to set the resting potential of certain excitatory cells, and may function in synaptic plasticity, and in the activation of sperm (Pape, (1996) Annu Rev Physiol 58: 299-327).

The pacemaker current Iₕ has unusual characteristics, including activation upon hyperpolarization, a tiny single-channel conductance, modulation by intracellular cyclic nucleotides, permeability to both K⁺ and Na⁺, and poor permeability to Li⁺. Iₕ is mediated by both Na⁺ (inward flux at a resting membrane potential near −70 mV) and K⁺ (outward flux at a resting membrane potential near −70 mV), and has a reversal potential around −30 or −40 mV under physiologic conditions (Ho et al. (1994), Pflugers Arch 426:68-74)(Mercuri et al., (1995) Eur J Neurosci 7: 462-9).

Four genes encoding ion channels that conduct pacemaker currents have recently been cloned. These genes belong to the HCN family, and have been designated as HCN1, HCN2, HCN3, and HCN4, respectively. HCN channels share structural features with voltage-gated K⁺ channels. These features include a GYG K⁺ channel signature sequence in the pore loop, and a highly positively charged S4 domain that is the putative voltage sensor (Gauss et al., (1998) Nature 393: 583-7; Ludwig et al., (1998) Nature 393: 587-91; Santoro et al., (1997) Proc Natl Acad Sci U S A 94: 14815-20; Santoro et al., (1998) Cell 93: 717-29). HCN channels are most homologous to the eag family of K⁺ channels (for example, erg, eag, elk) and the KAT1 family of plant K⁺ channels (Biel et al., (1999) Rev Physiol Biochem Pharmacol 136: 165-81) in that they possess six transmembrane domains, and incorporate an intracellular cyclic nucleotide binding domain that can modulate the voltage dependence of activation. For instance, binding of cAMP to HCN2 shifts the activation curve at least 20 mV to the right, thus enhancing channel activity at the resting membrane potential. These four HCN channels share substantial homology, but have different activation kinetics and degrees of responsiveness to cyclic AMP.

A significant feature of the increased spontaneous discharges observed in rodent neuropathic pain models is rhythmicity, whether rhythmic firing or rhythmic burst firing. This feature suggests underlying non-random processes for generation of the increased spontaneous discharges. In the present invention, we investigated the possible role of HCN pacemaker channels in neuropathic pain and other types of pain.

As used herein, a “HCN pacemaker channel” refers to a membrane channel, which is a hyperpolarization-activated, cyclic nucleotide-gated channel. A “HCN pacemaker channel” conducts both Na⁺ (inward flux from extracellular milieus to cytosol) and K⁺ (outward flux), and has a reversal potential around −30 or −40 mV under physiologic conditions. The single channel conductance for mammalian channels is thought to be quite low (Pape, (1996) Annu Rev Physiol 58: 299-327). An HCN pacemaker channel likely comprises tetramers of HCN pacemaker channel subunits. An HCN pacemaker channel may be heteromeric, when it is made of at least two different HCN pacemaker channel subunits, or homomeric, when it is made of the same HCN pacemaker channel protein subunits. An HCN pacemaker channel might also contain other subunits as accessories, such as Mirp1 (Yu et al., (2001) Circ Res 88: E84-7.).

As used herein, “HCN polypeptide” or “HCN subunit” refers to a polypeptide that is a subunit or monomer of a hyperpolarization-activated, cyclic nucleotide-modulated channel, a member of the HCN gene family. When an HCN polypeptide, e.g., HCN1, HCN2, HCN3, or HCN4, is part of an HCN pacemaker channel, either a homomeric or heteromeric potassium channel, the channel has hyperpolarization-activated, cyclic nucleotide-gated activity. The term HCN polypeptide therefore refers to polymorphic variants, alleles, mutants, and interspecies homologs that: (1) have a sequence that has greater than about 60% amino acid sequence identity, preferably about 65, 70, 75, 80, 85, 90, or 95% amino acid sequence identity, to an HCN pacemaker channel family member polypeptide such as human HCN1 (SEQ ID NO: 4), human HCN2 (GenBank Protein_Id: NP_{—}001185), human HCN3 (SEQ ID No: 10), and human HCN4 (GenBank Protein_Id: NP_{—}005468); (2) bind to antibodies, e.g., polyclonal or monoclonal antibodies, raised against an immunogen comprising an HCN pacemaker channel family member polypeptide, such as described above, and conservatively modified variants thereof; (3) encoded by a DNA molecule that specifically hybridizes under stringent hybridization conditions to a HCN pacemaker channel family member polynucleotide, such as human HCN1 (SEQ ID NO: 3), human HCN2 (GenBank Accession No: NM_{—}001194), human HCN3 (SEQ ID No: 9), and human HCN4 (GenBank Accession No: NM_{—}005477); or (4) encoded by a DNA molecule that can be amplified by primers that specifically hybridize under stringent hybridization conditions to an HCN pacemaker channel family member polynucleotide, such as described above.

Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

As used herein, a “HCN pacemaker gene” refers to a DNA molecule that (1) encodes a protein having a sequence that has greater than about 60% amino acid sequence identity, preferably about 65, 70, 75, 80, 85, 90, or 95% amino acid sequence identity, to an HCN pacemaker channel family member polypeptide such as human HCN1 (SEQ ID NO: 4), human HCN2 (GenBank Protein_Id: NP_{—}001185), human HCN3 (SEQ ID No: 10), and human HCN4 (GenBank Protein_Id: NP_{—}005468); (2) encodes a protein capable of binding to antibodies, e.g., polyclonal or monoclonal antibodies, raised against an immunogen comprising an HCN pacemaker channel family member polypeptide, such as described above, and conservatively modified variants thereof; (3) specifically hybridizes under stringent hybridization conditions to an HCN pacemaker channel family member polynucleotide, such as human HCN1 (SEQ ID NO: 3), human HCN2 (GenBank Accession No: NM_{—}001194), human HCN3 (SEQ ID No: 9), and human HCN4 (GenBank Accession No: NM_{—}005477); or (4) can be amplified by primers that specifically hybridize under stringent hybridization conditions to an HCN pacemaker family polynucleotide, such as described above.

As used herein, the term “HCN pacemaker channel family” is intended to mean two or more proteins or nucleic acid molecules having a common structural domain and having sufficient amino acid or nucleotide sequence identity to a known HCN pacemaker member, such as HCN1, HCN2, HCN3, or HCN4. Family members can be from either the same or different species. For example, a family can comprises two or more proteins of human origin, or can comprise one or more proteins of human origin and one or more of non-human origin.

In the present invention, we investigated levels of the mRNA and protein of HCN subunits, and whole cell current mediated by HCN pacemaker subunits in dorsal root ganglion (DRG) neurons from animal models of pain compared to those from the control animals.

As used herein, “control animal(s)” include a variety of preclinical animals that do not exhibit pain syndromes. “Animal models for pain” include a variety of preclinical animals that exhibit pain syndromes. Commonly studied rodent models of neuropathic pain include: the chronic constriction injury (CCI or Bennett) model; neuroma or axotomy models; the spinal nerve ligation (SNL or Chung) model; and the partial sciatic transection or Seltzer model(Shir et al., (1990) Neurosci Lett 115: 62-7). Neuropathic pain models include, but are not limited to, several traumatic nerve injury preparations (Bennett et al., (1988) Pain 33: 87-107; Decosterd et al., (2000) Pain 87: 149-58; Kim et al., (1992) Pain 50: 355-363; Shir et al., (1990) Neurosci Lett 115: 62-7), neuroinflammation models (Chacur et al., (2001) Pain 94: 231-44; Milligan et al., (2000) Brain Res 861: 105-16) diabetic neuropathy (Calcutt et al., (1997) Br J Pharmacol 122: 1478-82), virally induced neuropathy (Fleetwood-Walker et al., (1999) J Gen Virol 80: 2433-6.), vincristine neuropathy (Aley et al., (1996) Neuroscience 73: 259-65; Nozaki-Taguchi et al., (2001) Pain 93: 69-76.), and paclitaxel neuropathy (Cavaletti et al., (1995) Exp Neurol 133: 64-72). Commonly studied rodent models of inflammatory pain include: the complete Freund's adjuvant (CFA)-induced inflammation model, experimental burn injury models, the carrageenan inflammatory hyperalgesia model, the formalin test, and the rat inflamed knee and ankle joint models.

Assessment of pain and therapeutic responses to pharmacological and other interventions is done in a variety of ways, including behavioral and electrophysiological assessment, the latter providing “surrogate” outcomes. “Surrogate” assessments attempt to correlate physiological findings with behavior. Among the best-studied surrogate responses are electrophysiological responses of 1) primary afferent neurons, and 2) spinothalamic tract neurons in the dorsal horn of the spinal cord.

1. Two Full-length Human HCN Pacemaker Channel cDNAs Were Isolated and Characterized.

Two full-length human HCN pacemaker cDNA sequences (SEQ ID NO: 3 (hHCN1) and SEQ ID NO: 9 (hHCN3) were isolated and cloned from human spinal cord cDNA and human Marathon ready brain cDNA, respectively. The two DNA molecules encode two polypeptides, SEQ ID NO: 4 and SEQ ID NO: 10, respectively (Example 1).

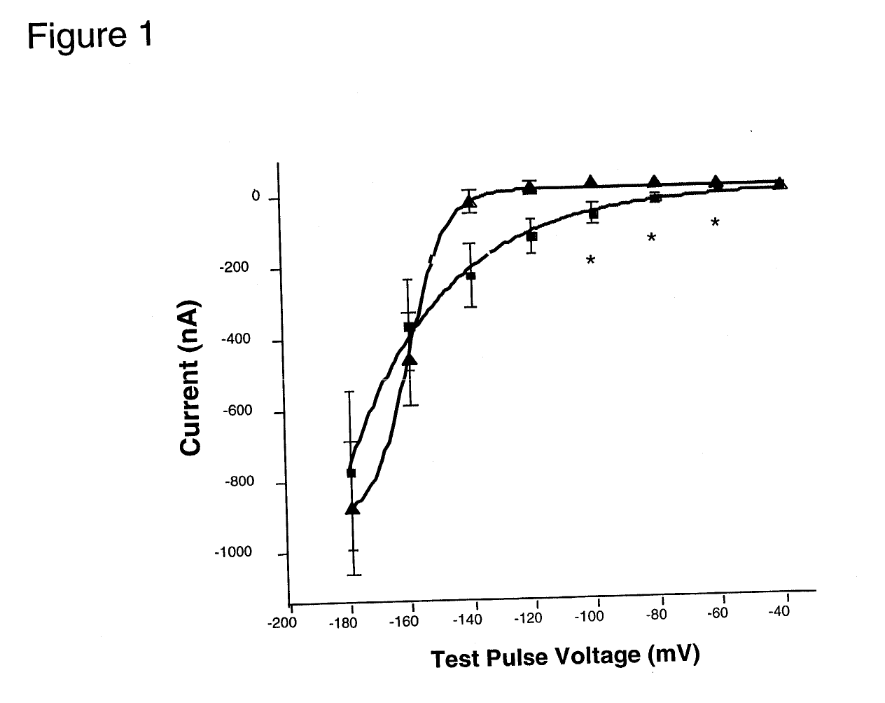

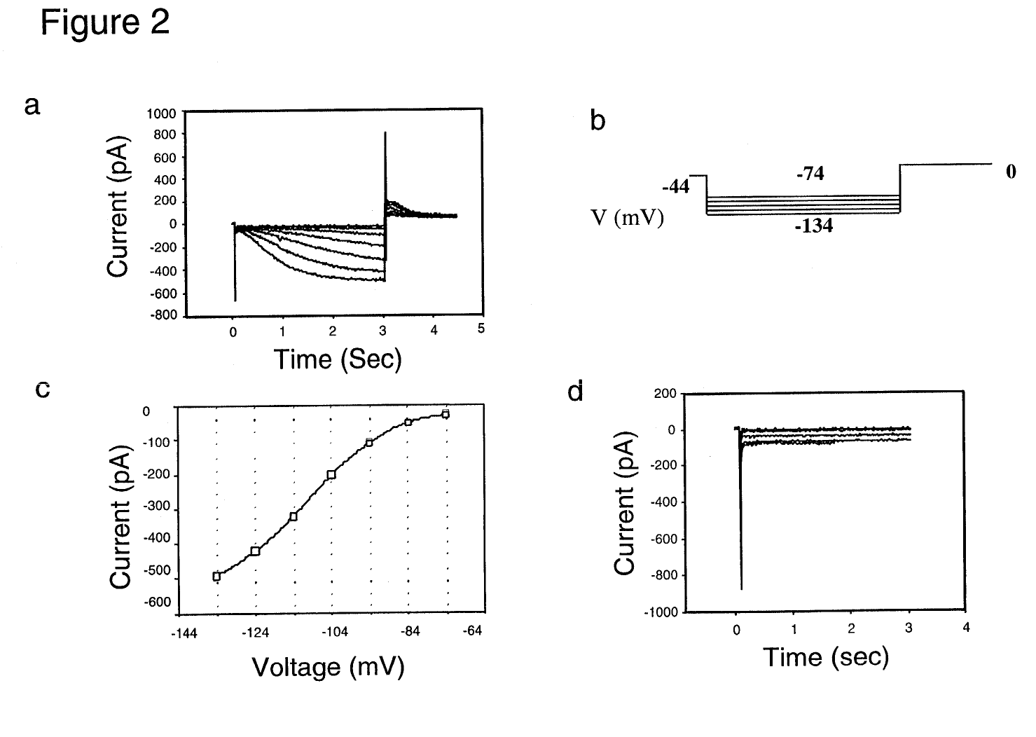

We have demonstrated that the two newly isolated full length human HCN pacemaker cDNA sequences encode protein products that form functional HCN pacemaker channels in either an oocyte expression system (FIG. 1 and Example 2) or a mammalian expression system (FIG. 2 and Example 3).

We subsequently found that similar sequences had been isolated and disclosed elsewhere (see Wo0063349, Wo0190142, Wo-202630, Wo0212340, and Wo9932615).

2. Specific Blockade of HCN Channels Suppressed Spontaneous Firing of Injured Primary Afferents and the Tactile Allodynia in an Animal Neuropathic Pain Model.

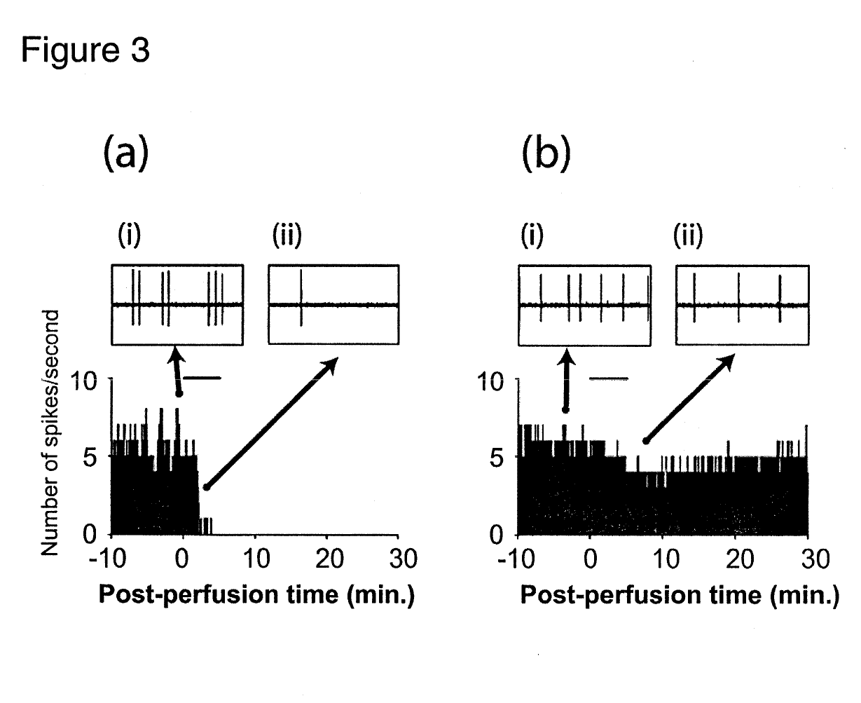

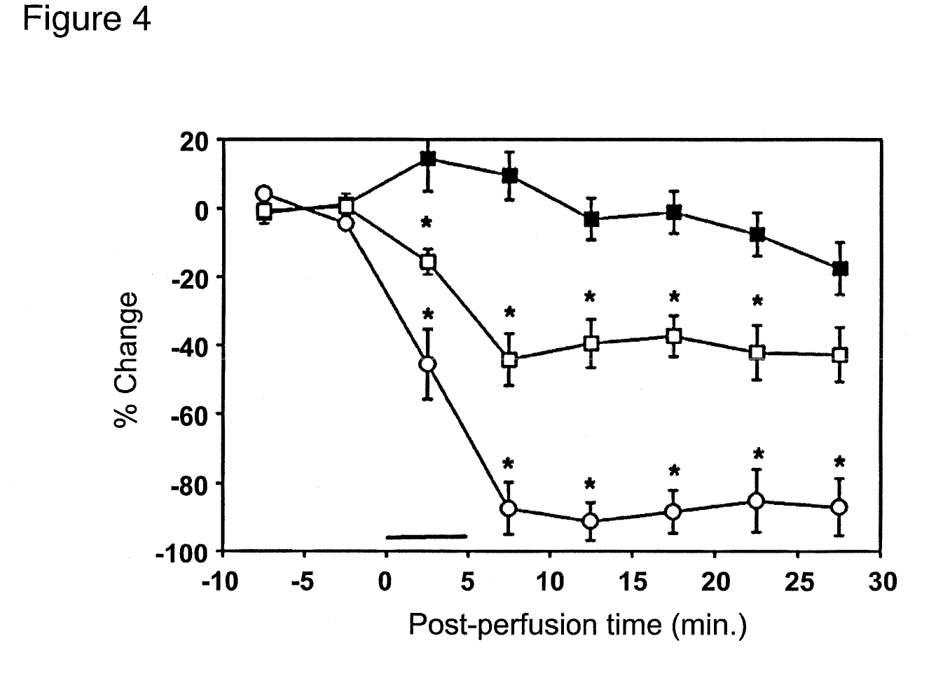

We performed extracellular recording in vitro on peripheral nerve fibers in control or previously ligated L4 or L5 (SNL) excised nerve-DRG preparations (Example 4). Spontaneous discharges arose from Aα/β neurons and some Aδ neurons (distinguished by their conduction velocity) in DRGs 1 to 3 weeks post injury (FIG. 3). Spontaneous action potentials tended to be rhythmic. Discharges from Aβ fibers were completely suppressed by bath application of 100 μM ZD7288, which is a specific blocker of Iₕ current (BoSmith et al., (1993) Br J Pharmacol 110: 343-9) but has no selectivity between HCN channel family members, for the duration of the extended period of observation (FIG. 3a, FIG. 4). Fiber identities (Aβ, A∂) were determined by evoked action potentials after data collection; in distinction to the suppression of spontaneous discharge, there was no conduction blockade of evoked action potentials observed after the application of ZD7288. Thus, these data illustrate that (1) Iₕ is critical to the generation of spontaneous firing of injured primary afferents, and that (2) blockade of Iₕ only suppresses spontaneous activity, but does not cause generalized failure of neuronal conduction (or nerve block).

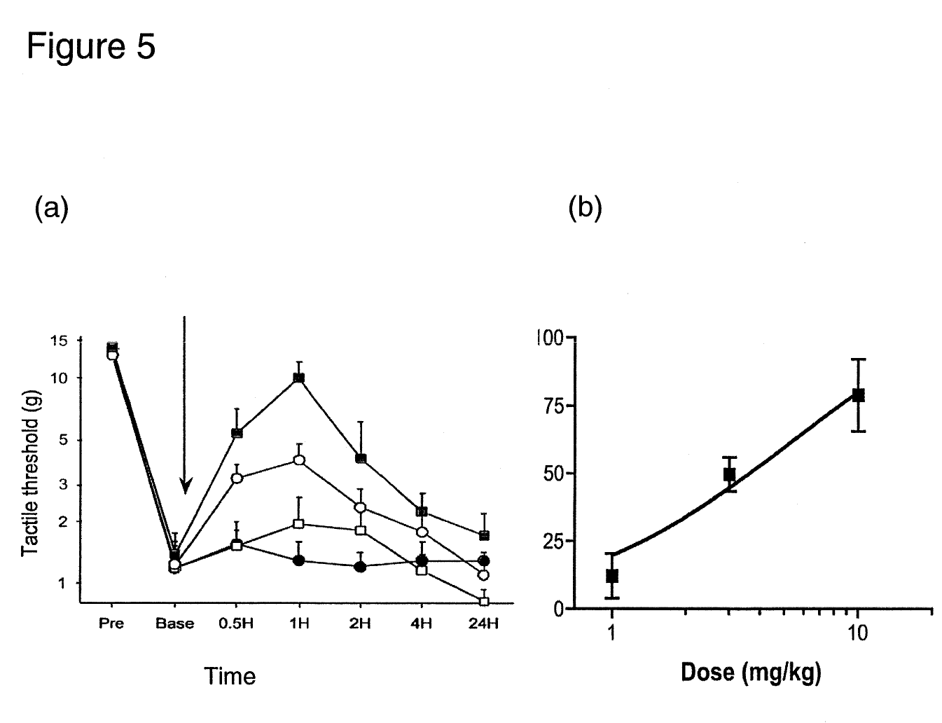

We also tested the role of Iₕ in animal behavioral studies. Allodynia, or pathological sensitivity to touch, is among the most troublesome of neuropathic symptoms, and is thought to arise from abnormal responses of large myelinated sensory fibers (Aβ) to stimulation. In the present invention, we used the SNL (unilateral L5/6 ligation) model to study the role of Iₕ in tactile allodynia (Example 5). We observed that ZD7288 (10 mg/kg) suppressed the tactile allodynia exhibited by awake SNL rats, without evidence of total sensory blockade or numbness and without overt adverse effects on behavior, in a dose-dependent manner (FIG. 5). Clearly, Iₕ contributes to pathologic neuronal activity manifested as tactile allodynia.

Results described supra demonstrated, for the first time, that Iₕ is critical to the generation of spontaneous firing of injured primary afferents, and that blockade of Iₕ ameliorates neuropathic pain behavior associated with such abnormal firing.

3. Specific Blockade of HCN Channels Did Not Yield Analgesia of a Clinically Relevant Magnitude Against Acute Thermal Stimuli.

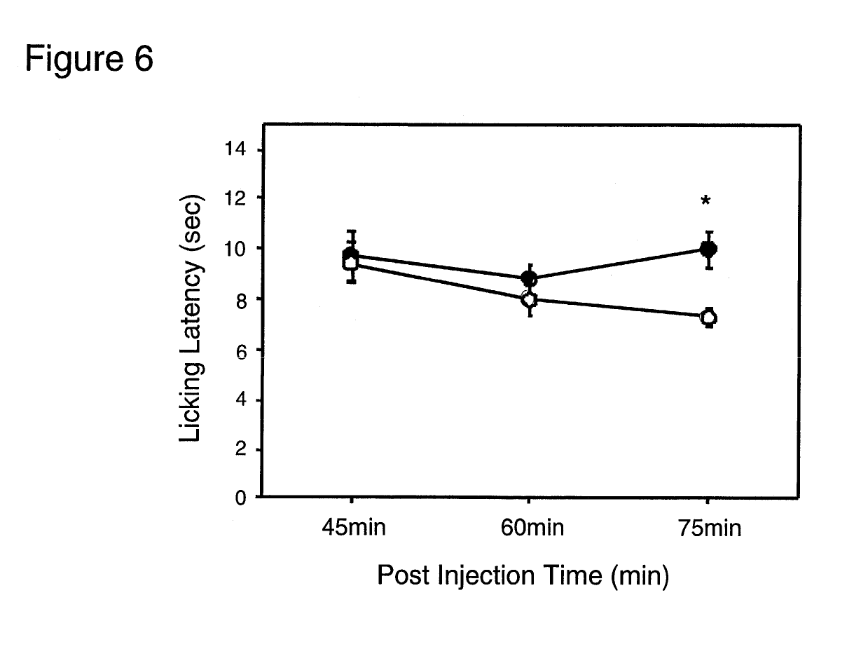

To test whether the antiallodynic effect seen with specific blockade of HCN channels, ZD7288, is a general analgesic effect independent of neuropathic pain, the hot plate test was performed to evaluate effects of ZD7288 on an acute thermally induced pain state with normal rats (Example 6). No statistically significant difference was seen between treatment with ZD7288 and saline at 45 or 60 min; a statistically significant, only minor difference was seen at 75 min (approximately 15%) (FIG. 6).

These results demonstrate that specific blockade of HCN channels does not yield analgesia of a clinically relevant magnitude against acute thermal stimuli. Therefore, the antiallodynic effects in the SNL model do not represent a generalized impairment of sensory function. In addition, these results demonstrate that ZD7288 does not impair the ability of rats to respond to perceived noxious stimuli; thus, the effect of ZD7288 on allodynia thresholds is not due to inhibition of motor responses or cognitive depression.

4. Specific Blockade of HCN Channels Suppressed the Tactile Allodynia in an Animal Inflammatory Pain Model.

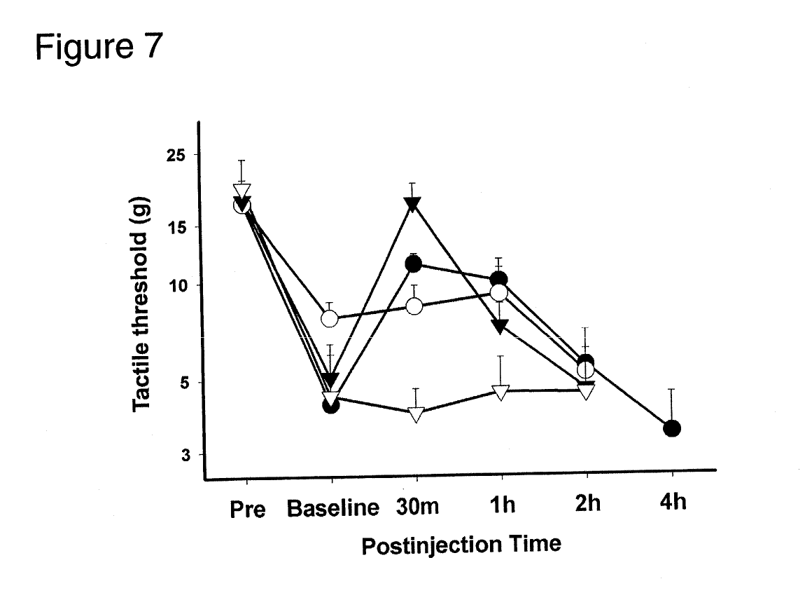

We also tested the role of HCN in inflammatory pain, which differs mechanistically and pharmacologically from neuropathic pain (Example 7). After injection of complete Freund's adjuvant (CFA) into one hind-paw of rats, animals developed marked tactile allodynia as measured using von Frey hairs (baseline, FIG. 7). Similar to morphine, a drug that is known to be effective against inflammatory pain, ZD7288 suppressed allodynia in CFA-injected rats, as shown by causing return of the paw threshold toward a normal, pre-CFA injection level (FIG. 7). Ibuprofen also showed some efficacy.

However, blockade of HCN channels with ZD7288 at 10 mg/kg, i.p., had no effect on thermal hyperalgesia (measured using a modified Hargreaves apparatus) in two different models of inflammatory pain: the rat carrageenan model of hindpaw inflammation, and the rat complete Freund's adjuvant (CFA) model of hindpaw inflammation. As shown supra, HCN blockade also had little effect on acute thermally evoked pain in the hot plate test.

Our data indicate that although specific blockade of HCN channels suppressed tactile allodynia in both neuropathic pain and inflammatory pain animal models, temperature sensation even in the presence of nociceptor sensitization in the periphery (e.g. skin) is not affected by Iₕ blockade. These results highlight the pharmacological differences between mechanical/tactile sensation and thermal perception, including thermal hyperalgesia, and appear consistent with our observation that the effect of ZD7288 is much more extensive on Aβ fibers (responsible for transducing mechanical/tactile sensation, not known to play a role in thermal sensation) than on A∂ fibers (responsible for transducing the “fast’ component of heat evoked pain). Our results suggest that specific blockade of HCN channels can be effective to suppress pain responding to mechanical stimuli in general.

5. Specific Blockade of HCN Channels Suppressed Spontaneous Pain in an Animal Model of Burn Injury Pain.

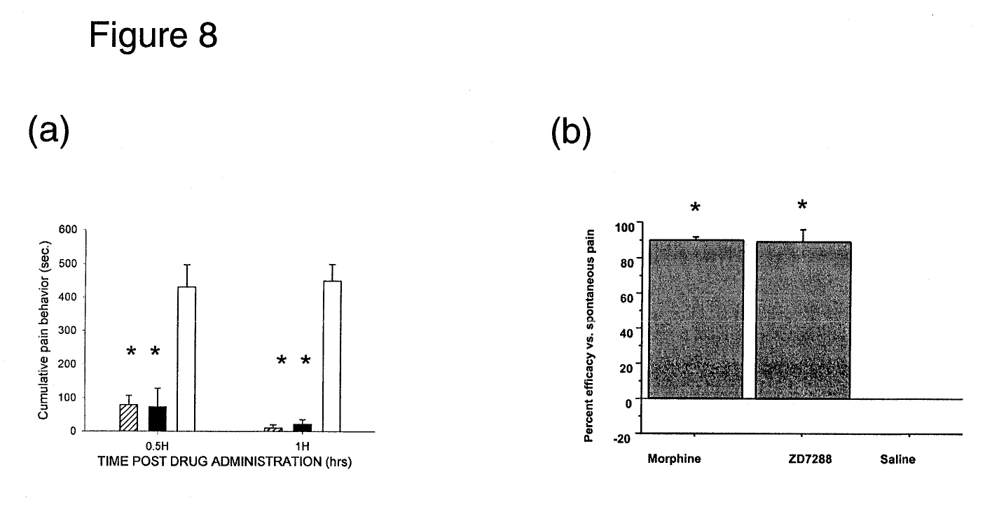

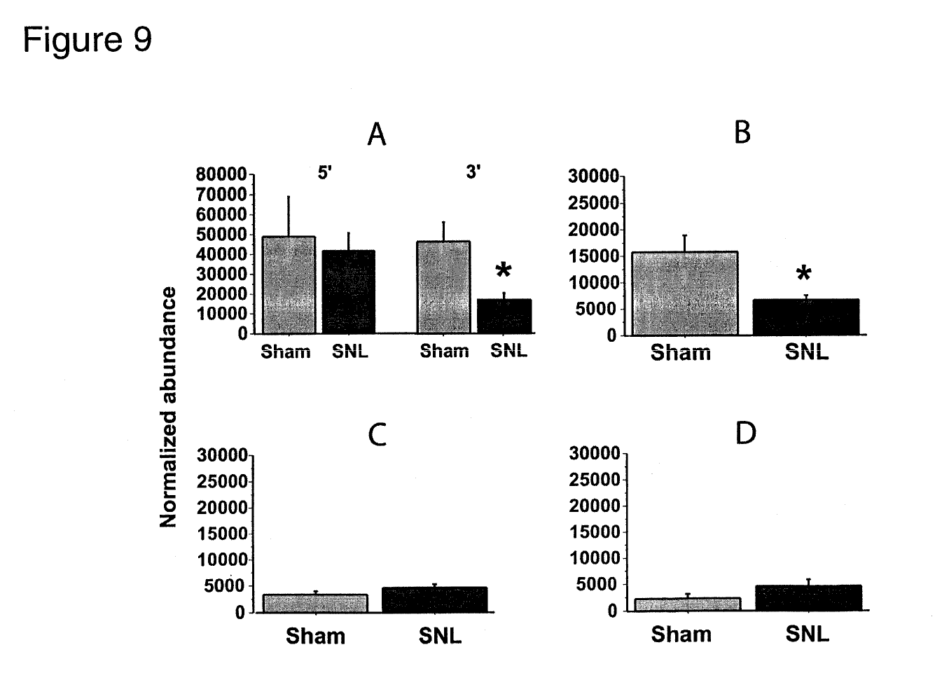

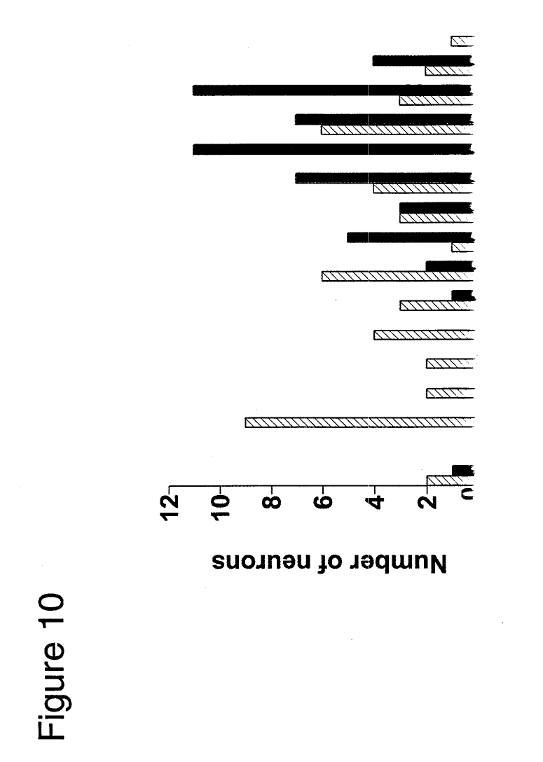

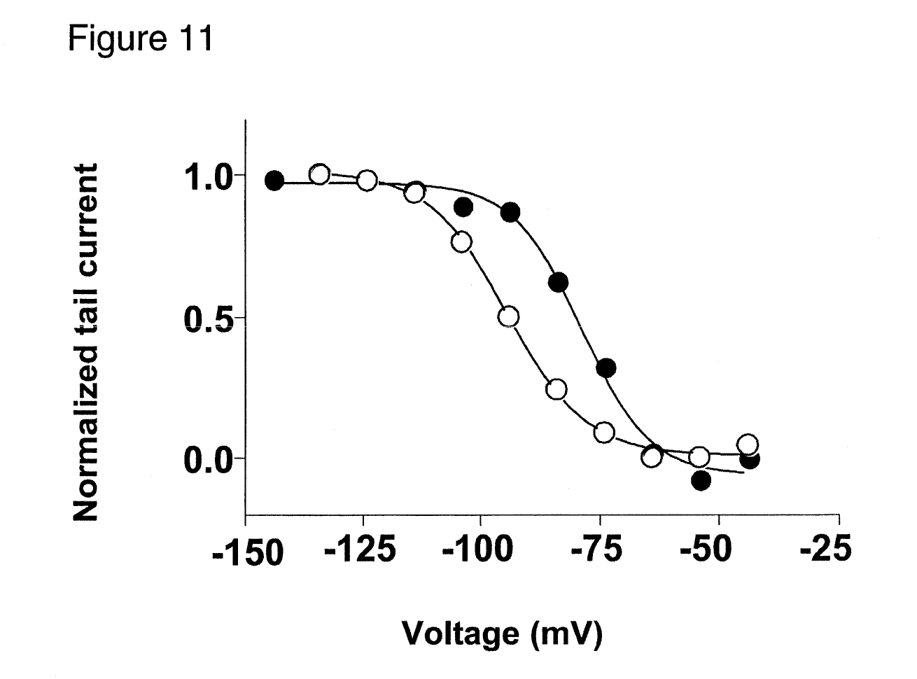

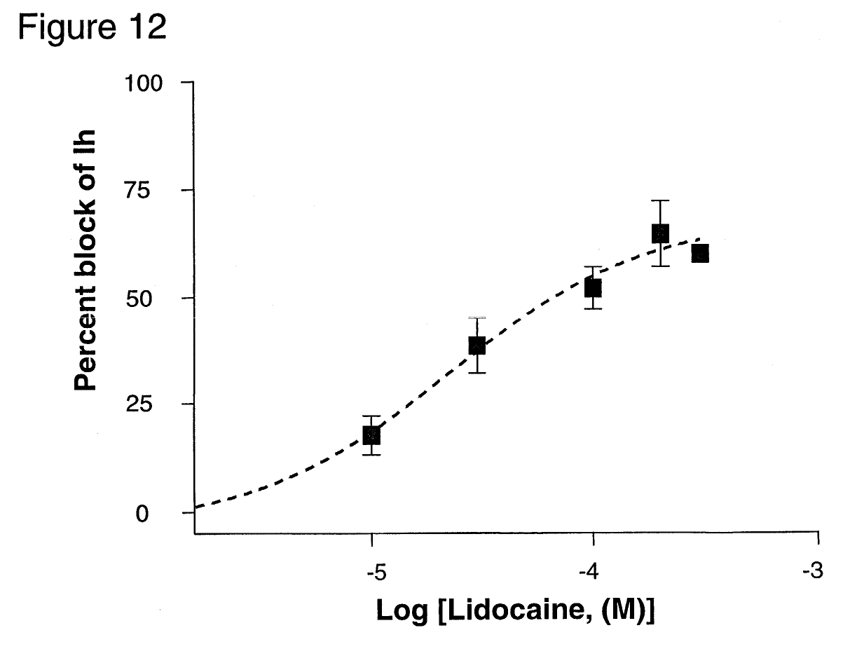

We further tested the role of HCN channels in spontaneous pain (Example 8). Both morphine and ZD7288 suppressed spontaneous pain in an animal model of burn injury pain (FIG. 8). No adverse behavioral effects were noted. Thus, Iₕ blockade is highly effective against spontaneous pain elicited by a first-degree burn injury.

Our data indicate that spontaneous, ongoing post-burn pain, which is experienced long after removal from the actual thermal contact, does not rely on the same transduction mechanisms as immediate thermal perception, and the two types of pain can be pharmacologically differentiated. Since post-burn pain is an obvious result of tissue damage, our results clearly suggest that tissue damage such as by a burn injury leads to the activation of resident HCN channels.

6. Specific Measurement of HCN mRNA or Protein Level.

The mRNA or protein level of a HCN in the DRG is measured by contacting the DRG with a compound or an agent capable of detecting the HCN mRNA or protein specifically.

A preferred agent for detecting HCN mRNA is a labeled nucleic acid probe capable of hybridizing specifically to the mRNA. For example, the nucleic acid probe specific for HCN1 mRNA can be, a full-length cDNA, such as the nucleic acid of SEQ ID NO: 3, or a portion thereof, such as an oligonucleotide of at least 15, 30, 50, 100, 250 or 500 nucleotides in length of SEQ ID NO: 3 and sufficient to hybridize to a HCN1 mRNA under stringent conditions. Preferably, a nucleic acid probe specific for HCN1 mRNA will only hybridize to HCN1 mRNA, not the mRNA of HCN2, HCN3, or HCN4 under stringent conditions.

A preferred agent for detecting a HCN protein is an antibody capable of binding specifically to the polypeptide, preferably a labeled antibody with a detectable label. Antibodies can be polyclonal or monoclonal. An intact antibody, or a fragment thereof (e.g., Fab or F(ab)₂) can be used.

The term “labeled”, with regard to the nuclei acid probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

HCN protein and mRNA in DRG can be assayed in vitro as well as in vivo. For example, in vitro techniques for detection of mRNA include Northern hybridizations, DNA microarray, and RT-PCR. In vitro techniques for detection of a polypeptide include enzyme linked immunosorbent assays (ELISAs), Western blots, immunoprecipitations and immunofluorescence. In vivo techniques for detection of mRNAs include transcriptional fusion described infra. In addition, as described in Example 9, HCN mRNA or proteins can also be assayed by in-situ hybridization and immunohistochemistry (to localized messenger RNA and protein to specific subcellular compartments and/or within neuropathological structures associated with the disease such as neurofibrillary tangles and amyloid plaques).

In addition, quantitative methods, such as positron emission tomography (PET) imaging make possible the assessment by noninvasive means of the changes of HCN proteins in the living human brain (Sedvall G, et al, (1988), Psychopharmacol Ser; 5:27-33). Tracer amounts of the HCN-binding radiotracers are injected intravenously into the subject, and the distribution of the radiolabeling in the brain of the subject can be imaged. Procedures for PET imaging are know to those skilled in the art.

7. Abnormal Function of HCN Pacemaker Channels in Sensory Neurons of a Neuropathic Pain Animal Model.

The present invention further demonstrated abnormal expression and activity of HCN pacemaker channels in sensory neurons of a neuropathic pain animal model.

First, quantitative real-time PCR (Example 9) comparison of RNA levels for the four HCN subtypes in whole L5/6 DRGs revealed that, in sham operated DRGs, the rank order abundance of transcripts was HCN1>>HCN2>HCN3, HCN4 (FIG. 9). These results differ slightly from the relative abundance described in murine whole DRG, where no HCN3 was detected. In the DRGs from nerve-ligated rats, we observed significant decreases in HCN1 mRNA using a primer pair directed toward the 3′ end of the coding sequence. Of note, no significant decrease was observed using a primer pair spanning a 5′ region containing intron #1 (Ludwig et al., (1999) Embo J 18: 2323-9). No significant changes were observed in mRNA for HCN3 and HCN4 (FIG. 9). In-situ hybridization using unique probes directed toward the 3′ end of the coding sequence showed that the decreases in HCN1 and HCN2 mRNA were generalized across all neurons and not confined to any specific neuronal subpopulation.

Second, immunohistochemical analysis (Example 9) showed that after nerve injury, changes in the amounts of detected HCN channel proteins mirrored changes seen in the amounts of HCN mRNA. Immunohistochemical staining of adjacent 10 μm sections revealed that HCN1, HCN2 and HCN3 were co-localized in the membrane region of predominantly, but not exclusively, larger neuronal profiles. Two different antibodies, directed toward either the N- or the C-terminus of HCN1, both revealed reduced membrane delineation in large neurons from nerve-ligated rats. The decrease in HCN1 immunoreactivity was quantified by Western blot: mean band density of HCN1 normalized to tubulin was lower, at 10.1+/−1.1 (dimensionless, +/−SEM) in injured DRGs in comparison to controls, at 16.3+/−1.7 (P<0.02, unpaired t-test). Marked decreases in HCN2 immunoreactivity were also apparent in injured DRGs compared to controls, again, in keeping with the PCR and in-situ data. While the distribution of HCN3 immunoreactivity suggested denser juxtamembranous staining in large neurons after injury, these changes were not clear enough to be considered definitive. No specific HCN4 immunoreactivity could be distinguished from background in either control or injured DRGs, likely due to low protein expression levels.

Third, whole cell patch clamp recordings (Example 10) from dissociated DRG neurons revealed a shift toward higher Iₕ current density in the nerve-ligated neurons. We compared Iₕ in single, acutely dissociated large neurons from the L5 DRGs of nerve-ligated (SNL) or sham-operated rats using the whole cell configuration of the patch clamp method. Nearly all large neurons (diameter 50±1 μm, mean±SEM) in both groups expressed currents consistent with Iₕ, as evidenced by their voltage and time dependent activation and their sensitivity to Cs⁺ (3 mM) and ZD7288 (50 μM). However, the distribution of current densities measured at −114 mV differed markedly between the two groups of neurons. A striking finding in SNL large L5 neurons was a shift toward a higher Iₕ current density distribution such that ˜92% expressed Iₕ greater than 4 pA/pF (FIG. 10, solid bars), compared with ˜42% of control neurons (FIG. 10, hatched bars). As used herein, the term “Iₕ current density” refers to the steady state inward current elicited by a voltage step normalized to the membrane capacitance, a measure of cell surface area. After nerve injury, the population of neurons having low Iₕ current density was significantly decreased, and the population of neurons expressing high current density was significantly increased (FIG. 10). This result is likely due to an increase in Iₕ expressed in injured neurons and not due to loss of a population of low expressing neurons since there is no evidence of DRG cell loss in the SNL injury model at this timepoint (Lekan et al., (1997) Neuroscience 81: 527-34). The cells from control and injured ganglia were indistinguishable with respect to cell size, and thus current density increases were due to an increase in expressed Iₕ and not a decrease in SNL neuron surface area as determined in dissociated ganglia preparations.

The observed shift toward higher Iₕ current density in the nerve-ligated neurons could be due to a number of parameters including an increase in open probability (pₒ) (e.g., as would result from a shift in the voltage dependence of Iₕ activation), an increase in the number of functional channels, or an increase in the current that a single channel fluxes. As used herein, the “activation threshold” refers to the voltage at which current is first detected. As used herein, “open probability (pₒ)” refers to the percentage of time that a channel is in the open conducting state.

Indeed, we found that the activation threshold of Iₕ was significantly more positive in DRG cells from the SNL rat compared to controls (Example 10 and FIG. 11). Furthermore, we found that the resting membrane potential was significantly more positive in SNL neurons, at −64.8±1.0 mV, (n=22), compared to controls, at −71.9±1.9 mV, (n=14; P<0.005), consistent with a larger contribution of Iₕ to the resting potential of SNL neurons. There was a tendency for the SNL DRG neurons to have faster kinetics of activation when activated by voltage steps to less than −100 mV (FIG. 11). This difference is likely related to the shift in threshold for activation of Iₕ to more depolarized values.

8. Iₕ is Blocked by Lidocaine.

Systemically administered lidocaine has been known to be a useful treatment for neuropathic pain for some time (for review, see [Chaplan, (1997) Anesthesia: Biologic Foundations (eds. Biebuyck, J. et al.) Raven Press, New York]. When administered systemically so as to attain plasma drug concentrations within the range considered safe and therapeutic against cardiac dysrhythmias, lidocaine shows specific anti-hyperalgesic activity in neuropathic pain states, whereas it does not appear to be useful as a general analgesic in experimental or clinical acute pain states. The anti-hyperalgesic activity occurs selectively without blockade of normal sensory function; specifically, the concentrations required for this effect are very much below the concentrations necessary to attain conduction blockade of peripheral nerves.

These same selective anti-hyperalgesic effects are demonstrable in preclinical models of neuropathic pain (again, see Chaplan, 199.7 supra; also Abram et al., (1994) Anesthesiology 80: 383-391; Chaplan et al., (1995) Anesthesiology 83: 775-785). However, anti-hyperalgesic effects are not a general property of sodium channel blocking compounds in preclinical models: for example, bupivacaine, which is structurally similar to lidocaine, does not possess any anti-hyperalgesic activity [Chaplan (1999) Opioid sensitivity of chronic non-cancer pain (eds. E., K. & Wiesenfeld-Hallin, Z.) (IASP Press)]. In these same models, it has been amply demonstrated that systemically administered lidocaine also stops the ectopic firing in injured peripheral nerves (Devor et al., (1992) Pain 48: 261-268), in a manner similar to the data shown here for ZD7288.

Since lidocaine is generally considered a sodium channel blocker, the mechanistic basis of the antihyperalgesic effect has until now been attributed to sodium channel blockade. The present invention has shown that lidocaine blocks Iₕ in acutely dissociated rat dorsal root ganglion neurons, in a concentration dependent manner (Example 12 and FIG. 12). This blockade occurs over a concentration range that is approximately similar to the range in which lidocaine blocks sodium channels(Gold et al., (2001) J Pharmacol Exp Ther 299: 705-11.). Lidocaine has previously been reported to block Iₕ in a different preparation, the rabbit sinoatrial node(Rocchetti et al., (1999) J Cardiovasc Pharmacol 34: 434-9.); the ED50 of 38.2 micromolar previously reported is comparable to the ED50 of 23 micromolar described here. Similarly, QX-314, the extracellularly restricted quaternary amide analog of lidocaine, blocks Iₕ when applied intracellularly at 5 or 10 mM (Perkins et al., (1995) J Neurophysiol 73: 911-5).

Thus, the therapeutic effect of systemically administered lidocaine in neuropathic pain may reside wholly or in part in blockade of HCN channels by lidocaine rather than sodium channel blockade. This provides additional demonstration, with examples in the clinical literature, of the potential for utility of compounds directed at HCN channels in neuropathic pain, and in addition provides demonstration of another Iₕ blocking compound identified by our screening techniques.

The present invention demonstrated for the first time that abnormal function of HCN pacemaker channels contributes importantly to spontaneous electrical behavior and abnormal resting membrane potential after painful nerve injury. Specific blockade of HCN channels suppressed pain resulting from nerve injury, inflammation and mechanical stimulation, as well as spontaneous pain. The effects of HCN blockade appear to be sensory-modality specific, as opposed to model specific. For example, in the same inflammatory CFA pain model, specific pharmacological blockade of HCN channels by administering ZD7288 to the animal had no effect on thermal hyperalgesia but markedly suppressed tactile allodynia. The modalities most affected are spontaneous pain and tactile allodynia, which are the two most troublesome complaints of patients with neuropathic pain in clinical studies. This observation has important implications for the ability of a pharmacological treatment to selectively stop pain, without causing a generalized loss of normal sensation.

The present invention provides an entirely new synthesis of the pathophysiology governing pain syndromes in general, enabling directed research toward useful interventions to prevent or treat these disorders. By analogy, insights gained from neuronal dysregulation leading to spontaneous activity manifested as pain, can illuminate other disorders involving ectopic or excessive spontaneous electrical activity or dysregulation of spontaneous activity, including but not limited to epileptiform disorders, psychiatric illnesses, cardiac arrhythmias, tinnitus, Tourette's syndrome, hemiballismus, choreoathetosis, sleep apneas, sudden infant death syndrome, irritable bowel syndrome, and restless leg syndrome.

Antibody That Specifically Binds the Carboxy-terminus of a HCN Protein

The present invention encompasses antibodies that specifically bind the carboxy(C)-terminus of a HCN protein. The term “antibody” as used herein refers to immunoglobulin molecules and immunologically active portions of immunoglobulin molecules, i.e., molecules that contain an antigen binding site which specifically binds an antigen, such as the C-terminus of a HCN polypeptide. A molecule which specifically binds an antigen, binds only the antigen, but does not substantially binds other molecules in a sample, e.g., a biological sample, which naturally contains the antigen polypeptide. Examples of immunologically active portions of immunoglobulin molecules include Fab and F(ab)₂ fragments which can be generated by treating the antibody with an enzyme such as pepsin.

In various embodiments, the substantially purified antibodies of the invention, or fragments thereof, can be human, non-human, chimeric and/or humanized antibodies. Such antibodies of the invention can be, but are not limited to, goat, mouse, rat, sheep, horse, chicken, or rabbit antibodies. In addition, such antibodies of the invention can be polyclonal antibodies or monoclonal antibodies. The term “monoclonal antibody” or “monoclonal antibody composition”, as used herein, refers to a population of antibody molecules that contain only one species of an antigen-binding site capable of immunoreacting with a particular epitope. The term “polyclonal antibody” refers to antibodies directed against a polypeptide or polypeptides of the invention capable of immunoreacting with more than one epitope. Particularly preferred polyclonal antibody preparations are ones that contain only antibodies directed against a polypeptide or polypeptides of the invention.

The term “antigen” as used herein refers to a molecule containing one or more epitopes that will stimulate a host's immune system to make a humoral and/or cellular antigen-specific response. The term is also used herein interchangeably with “immunogen”.

The term “epitope” as used herein refers to the site on an antigen or hapten to which a specific antibody molecule binds. The term is also used herein interchangeably with “antigenic determinant” or “antigenic determinant site.”

The term “the carboxy-terminus of a HCN protein” or “the C-terminus of a HCN protein” as used herein refers to the fragment of a HCN protein which comprises the end of the HCN protein having a free carboxyl (—COOH) group, but does not include the six transmembrane segments of the HCN protein. Examples of the C-terminus of a HCN protein can be the linker region between the last transmembrane segment and the cyclic nucleotide-binding domain (CNBD), the CNBD, the extreme C-terminus including the last 50 amino acid residues of the HCN, or the combination thereof.

An isolated C-terminus of a HCN protein can be used as an immunogen to generate antibodies using standard techniques for polyclonal and monoclonal antibody preparation. The immunogen comprises at least 8 (preferably 20, 30, or more) amino acid residues of the C-terminus of a HCN protein and encompasses an epitope of the protein such that an antibody raised against the peptide forms a specific immune complex with the protein. Preferred epitopes encompassed by the antigenic peptide are regions that are located on the surface of the protein, e.g., hydrophilic regions of the proteins of the invention. Hydrophobic or hydrophilic regions on a protein can be identified using hydrophobicity plotting software programs. The immunogen can be obtained using protein expression and isolation techniques known to those skilled in the art, such as recombinant expression from a host cell, chemical synthesis of proteins, or in vitro transcription/translation. Particularly preferred immunogen compositions are those that contain no other animal proteins such as, for example, immunogen recombinantly expressed from a non-animal host cell, i.e., a bacterial host cell.

Polyclonal antibodies can be raised by immunizing suitable subject animals such as mice, rats, guinea pigs, rabbits, goats, horses and the like, with rabbits being preferred. Preimmune serum is collected prior to the first immunization. Each animal receives between about 0.001 mg and about 1000 mg of the immunogen either with or without an immune adjuvant. Acceptable adjuvants include, but are not limited to, Freund's complete, Freund's incomplete, alum-precipitate, water in oil emulsion containing Corynebacterium parvum and tRNA. The initial immunization consists of the polypeptide in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three-week intervals until maximal titers are obtained. At about 7 days after each booster immunization or about weekly after a single immunization, the animals are bled, the serum collected, and aliquots are stored at about −20° C.

Monoclonal antibodies (mAb) are prepared by immunizing inbred mice, preferably Balb/c, with the immunogen. The mice are immunized by the IP or SC route with about 0.001 mg to about 1.0 mg, preferably about 0.1 mg, of HCN C-terminus polypeptide in about 0.1 ml buffer or saline incorporated in an equal volume of an acceptable adjuvant, as discussed above. Freund's adjuvant is preferred, with Freund's complete adjuvant being used for the initial immunization and Freund's incomplete adjuvant used thereafter. The mice receive an initial immunization on day 0 and are rested for about 2 to about 30 weeks. Immunized mice are given one or more booster immunizations of about 0.001 to about 1.0 mg of the immunogen in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions that will allow the formation of stable hybridomas. Fusion partners may include, but are not limited to: mouse myelomas P3/NS1/Ag 4-1; MPC-11; S-194 and Sp2/0, with Sp2/0 being generally preferred. The antibody producing cells and myeloma cells are fused in polyethylene glycol, about 1000 mol. wt., at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using polypeptide of the invention as the antigen. The culture fluids are also tested in the Ouchterlony precipitation assay to determine the isotype of the mAb. Hybridoma cells from antibody positive wells are cloned by a technique such as the soft agar technique of MacPherson (Soft Agar Techniques, in Tissue Culture Methods and Applications, Kruse and Paterson, Eds., Academic Press, 1973 or by the technique of limited dilution).

Monoclonal antibodies can be produced in vivo by injection of pristane primed Balb/c mice, approximately 0.5 ml per mouse, with about 1×106 to about 6×106 hybridoma cells at least about 4 days after priming. Ascites fluid is collected at approximately 8-12 days after cell transfer and the monoclonal antibodies are purified by techniques known in the art.

Monoclonal Ab can also be produced in vitro by growing the hydridoma in tissue culture media well known in the art. High density in vitro cell culture may be conducted to produce large quantities of mAbs using hollow fiber culture techniques, air lift reactors, roller bottle, or spinner flasks culture techniques well known in the art. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. The antibody molecules can be isolated from the mammal (e.g., from the blood) or culture cells and further purified by well-known techniques, such as protein A chromatography to obtain the IgG fraction. Alternatively, can be selected for (e.g., partially purified) or purified by, e.g., affinity chromatography. For example, a recombinantly expressed and purified (or partially purified) immunogen of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the immunogen of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired immunogen of the invention.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region (See, e.g., U.S. Pat. No. 4,816,567; and U.S. Pat. No. 4,816,397). Humanized antibodies are antibody molecules from non-human species having one or more complementarily determining regions (CDRs) from the non-human species and a frame work region from a human immunoglobulin molecule (See, e.g., U.S. Pat. No. 5,585,089). Such chimeric and humanized monoclonal antibodies can be produced by recombinant DNA techniques known in the art, for example using methods described in PCT Publication No. WO 87/02671; European Patent Application 184,187; PCT Publication No. WO 86/01533; and U.S. Pat. No. 4,816,567.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., the immunogen of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar ((1995), Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

An antibody directed against an HCN can be used to isolate the HCN polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. A detectable substance can be coupled to the antibody to facilitate protein detection. Such detectable substance can be various enzymes, prosthetic groups, fluorescent materials, luminescent materials, bioluminescent materials, or radioactive materials. Examples of suitable enzymes include horseradish peroxidase, alkaline phosphatase, beta-galactosidase, or acetyleholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵1, ¹³¹1, ³⁵S or ³H.

Further, an antibody (or fragment thereof) of the invention can be conjugated to a therapeutic moiety such as a therapeutic agent or a radioactive metal ion for modifying a given biological response, such as inhibiting the conductance of current through a HCN channel. The therapeutic moiety is not to be construed as limited to classical chemical therapeutic agents. For example, the drug moiety may be a protein or polynucleotide possessing a desired biological activity. Techniques for conjugating such therapeutic moiety to antibodies are well known, see, e.g., Amon et al., (1985), Monoclonal Antibodies And Cancer Therapy, Reisfeld et al. (eds.), pp. 243-56 (Alan R. Liss, Inc.); Hellstrom et al., (1987), Controlled Drug Delivery (2nd Ed.), Robinson et al. (eds.), pp. 623-53 (Marcel Dekker, Inc.); and Thorpe et al., (1982), Immunol. Rev., 62:119-58.

Method of Reducing Pain by Targeting HCN Pacemaker Channels

The present invention provides new methods for reducing pain, preferably neuropathic pain or inflammatory pain, by targeting HCN pacemaker channels.

The term “pain” as used herein refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischemia, and diabetic neuropathy (see, e.g., Harrison's Principles of Internal Medicine, pp. 93-98 (Wilson et al., eds., 12th ed. 199 1); Williams et al., (1999) J of Medicinal Chem. 42:1481-1485), herein each incorporated by reference in their entirety).

As used herein “neuropathic pain” refers to pain induced by injury or disease of the peripheral or central sensory pathways, where the pain often occurs or persists without an obvious noxious input. It is selected from the group consisting of carpal tunnel syndrome, central pain, complex regional pain syndrome (CRPS), diabetic neuropathy, opioid resistant pain, phantom limb pain, postmastectomy pain, thalamic syndrome (anesthesia dolorosa), lumbar radiculopathy; cancer related neuropathy, herpetic neuralgia, HIV related neuropathy, multiple sclerosis, and pain caused by immunologic mechanisms, multiple neurotransmitter system dysfunction, nervous system focal ischemia, and neurotoxicity.

As used herein “inflammatory pain” refers to pain induced by inflammation. Such types of pain may be acute or chronic and can be due to any number of conditions characterized by inflammation including, without limitation, sunburn, rheumatoid arthritis, osteoarthritis, colitis, carditis, dermatitis, myositis, neuritis and collagen vascular diseases.

The term “subject” as used herein, refers to an animal, preferably a mammal, most preferably a human, who has been the object of treatment, observation or experiment.

The term “control individual” as used herein, refers to the same animal as that of the subject to whom it compares with, who has no pain syndromes.

The term “prophylactically effective dose” refers to that amount of active compound or pharmaceutical agent that inhibits in a subject the onset of a pain as being sought by a researcher, veterinarian, medical doctor or other clinician, the delaying of the pain is mediated by the modulation of an HCN pacemaker channel activity. Methods are known in the art for determining the prophylactically effective dose of an active compound or pharmaceutical agent.

In another aspect, the present invention relates to a method for treating pain, preferably neuropathic pain or inflammatory pain, in a subject in need thereof, comprising administering to the subject a therapeutically effective dose of a composition that decreases the current mediated by an HCN pacemaker channel in a sensory cell of the subject.

The invention further provides a combination therapy for preventing the onset of or treating pain, preferably neuropathic pain or inflammatory pain, in a subject in need of, by administering to the subject a prophylactically or therapeutically effective dose of a composition that decreases the current mediated by an HCN pacemaker channel in a sensory cell of the subject, in combination with one or more other analgesics or adjuvants, such as morphine or other opiate receptor agonists; nalbuphine or other mixed opioid agonist/antagonists; tramadol; baclofen; clonidine or other alpha-2 adrenoreceptor agonists; amitriptyline or other tricyclic antidepressants; gabapentin or pregabalin, carbamazepine, phenytoin, lamotrigine, or other anticonvulsants; and/or lidocaine, tocainide, or other local anesthetics/antiarrhythmics.

The term “therapeutically effective dose” refers to that amount of an active composition alone, or together with other analgesics, that produces the desired reduction of pain. In the case of treating a condition characterized by a higher current density of Iₕ from the sensory neurons of the subject, the desired reduction of pain is associated with decreased current density of Iₕ from the sensory neurons of the subject to a level that is within a normal range found in a control individual not suffering from pain.

As used herein, the term “composition” is intended to encompass a product comprising the specified ingredients in the specified amounts, as well as any product which results, directly or indirectly, from combinations of the specified ingredients in the specified amounts, provided that the specified ingredients in the specified amounts have not been previously used in a method for treating pain, preferably neuropathic pain or inflammatory pain, in a subject in need thereof. For example, the term “composition” as used herein shall not include compounds lidocaine, clonidine, and any other inhibitor for HCNs that have been used in a method for treating pain previously. The term “inhibitor” for HCN is as defined infra.

In one embodiment, the present invention provides a method for treating pain, preferably neuropathic pain or inflammatory pain, in a subject in need thereof, by administering to the subject a therapeutically effective dose of a composition that decreases the open probability of HCN channels, for example by blocking the pore, stabilizing non-conducting states, or by shifting the voltage dependence of Iₕ activation in the sensory cells of the subject. Preferably, the composition blocks current flux through the channel or shifts the activation threshold of HCN pacemaker channels in sensory neurons toward more negative potentials. One example of such compounds is ZD7288; others can be identified by methods described infra.

In another embodiment, the present invention provides a method for treating pain, preferably neuropathic pain or inflammatory pain, in a subject in need thereof, by administering to the subject a therapeutically effective dose of a composition that decreases ion conductance of HCN channels in sensory cells of the subject. Examples of such compositions include but are not limited to, ZD7288, ZM-227189 (Astra Zeneca), Zatebradine, DK-AH268, alinidine (Boehringer Ingelheim), ivabradine (Servier). More compounds that decrease single HCN channel conductance can be identified using methods described infra.

In yet another embodiment, the present invention provides a method for treating a pain, preferably a neuropathic pain or inflammatory pain in a subject in need thereof, by administering to the subject a therapeutically effective dose of a composition that decreases the number of functional HCN channels in sensory cells of the subject. Preferably, the method involves a composition that decreases the expression of HCN pacemaker proteins, in sensory cells of the subject. Examples of such a composition include compounds that repress HCN transcription or translation, which can be identified by methods described infra. In addition, antisense nucleic acids or small interfering RNAs (siRNAs) can also be used to reduce the expression of HCN pacemaker proteins through gene therapy.

The invention is amenable to antisense nucleic acids or siRNA based strategies by reducing expression of HCN pacemaker proteins in sensory cells of a subject. The principle of antisense nucleic acids strategies is based on the hypothesis that sequence-specific suppression of gene expression can be achieved by intracellular hybridization between mRNA and a complementary antisense species. The formation of a hybrid RNA duplex may then interfere with the processing/transport/translation and/or stability of the target HCN mRNA. Hybridization is required for the antisense effect to occur. Antisense strategies may use a variety of approaches including the use of antisense oligonucleotides, injection of antisense RNA and transfection of antisense RNA expression vectors. Phenotypic effects induced by antisense effects are based on changes in criteria such as protein levels, protein activity measurement, and target mRNA levels.

An antisense nucleic acid can be complementary to an entire coding strand of an HCN pacemaker gene, or to only a portion thereof. An antisense nucleic acid molecule can also be complementary to all or part of a non-coding region of the coding strand of an HCN pacemaker gene. The non-coding regions (“5′ and 3′ untranslated regions”) are the 5′ and 3′ sequences that flank the coding region and are not translated into amino acids. Preferably, the non-coding region is a regulatory region for the transcription or translation of the HCN pacemaker channel gene. The term “regulatory region” or “regulatory sequence” is intended to include promoters, enhancers and other expression control elements (e.g., polyadenylation signals, and ribosome binding site (for bacterial expression) and, an operator). Such regulatory sequences are described and can be readily determined using a variety of methods known to those skilled in the art (see for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences).

An antisense oligonucleotide of the invention can be, for example, a length of about 15, 20, 25, 30, 35, 40, 45 or 50 nucleotides or more that is complementary to the nucleotide sequence of human HCN 1 (SEQ ID NO:3), human HCN2 (GenBank Accession No: NM_{—}001194), human HCN3 (SEQ ID NO:9), or human HCN4 (GenBank Accession No:NM_{—}005477). An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides that can be used to generate the antisense nucleic acid include 5-fluorouracil, 5-bromouracil, 5-chlorouracil, 5-iodouracil, hypoxanthine, xanthine, 4-acetylcytosine, 5- (carboxyhydroxylmethyl) uracil, 5-carboxytnethylaminomethyl-2-thiouridine, 5-carboxymethylaminomethyluracil, dihydrouracil, beta-D-galactosylqueosine, inosine, N6-isopentenyladenine, I-methylguanine, 1-methylinosine, 2,2-dimethylguanine, 2-methyladenine, 2- methylguanine, 3-methyleytosine, 5-methylcytosine, N6-adenine, 7- methylguanine, 5-methylaminomethyluracil, 5-methoxyaminomethyl-2-thiouracil, beta-D- mannosylqueosine, 5′-methoxycarboxymethyluracil, 5-methoxyuracil, 2- methylthio-N6- isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5- methyluracil, uracil-5- oxyacetic acid methylester, uracil-5-oxyacetic acid (v), 5-methyl-2- thiouracil, 3-(3- amino-3-N-2-carboxypropyl) uracil, (acp3 )w, and 2,6-diaminopurine. An antisense nucleic acid molecule can be a CC-anomeric nucleic acid molecule. A CC-anomeric nucleic acid molecule forms specific double-stranded hybrids with complementary RNA in which, the strands run parallel to each other (Gaultier et al. (1987) Nucleic Acids Res. 15:6625-664 1). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Alternatively, the antisense nucleic acid can also be produced biologically using an expression vector into which a nucleic acid has been subcloned in an antisense orientation (i.e., RNA transcribed from the inserted nucleic acid will be of an antisense orientation to a target nucleic acid of interest). That is, a DNA molecule is operably linked to a regulatory sequence in a manner that allows for expression (by transcription of the DNA molecule) of an RNA molecule that is antisense to the mRNA encoding an HCN pacemaker protein. Regulatory sequences operably linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. ((1986), Reviews—Trends in Genetics, Vol. 1(1)).

The antisense nucleic acid molecules of the invention are typically administered to a subject or generated in situ such that they hybridize with or bind to cellular mRNA and/or genomic DNA encoding an HCN protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. Antisense nucleic acid molecules can be administered to the subject via direct injection or surgical implantation in the proximity of the damaged tissues or cells in order to circumvent their exclusion from the central nervous system (CNS) by an intact blood-brain barrier. Successful delivery of nucleic acid molecules to the CNS by direct injection or implantation has been documented (See e.g., Otto et al., (1989), J. Neurosci. Res. 22: 83-91; Goodman & Gilman's The Pharmacological Basis of Therapeutics, 6th ed, pp 244; Williams et al., (1986), Proc. Natl. Acad. Sci. USA 83: 9231-9235; and Oritz et al., (1990), Soc. Neurosci. Abs. 386: 18).

Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens.

The antisense nucleic acid molecules can also be generated in situ by expression from vectors described herein harboring the antisense sequence. To achieve sufficient intracellular concentrations of the antisense molecules, vector constructs in which the antisense nucleic acid molecule is placed under the control of a strong pol II or pol III promoter are preferred.

In a preferred embodiment, the method of treating a pain in a subject in need thereof involves the use of small interfering RNA (siRNA). In several organisms, introduction of double-stranded RNA has proven to be a powerful tool to suppress gene expression through a process known as RNA interference. Many organisms possess mechanisms to silence any gene when double-stranded RNA (dsRNA) corresponding to the gene is present in the cell. The technique of using dsRNA to reduce the activity of a specific gene was first developed using the worm C. elegans and has been termed RNA interference, or RNAi (Fire, et al., (1998), Nature 391: 806-811). RNAi has since been found to be useful in many organisms, and recently has been extended to mammalian cells in culture (see review by Moss, (2001), Curr Biol 11: R772-5).

An important advance was made when RNAi was shown to involve the generation of small RNAs of 21-25 nucleotides (Hammond et al., (2000) Nature 404: 293-6; Zamore et al., (2000) Cell 101: 25-33). These small interfering RNAs, or siRNAs, may initially be derived from a larger dsRNA that begins the process, and are complementary to the target RNA that is eventually degraded. The siRNAs are themselves double-stranded with short overhangs at each end; they act as guide RNAs, directing a single cleavage of the target in the region of complementarity (Elbashir et al., (2001) Genes Dev 15: 188-200; Zamore et al., (2000) Cell 101: 25-33).

Methods of producing siRNA, 21-23 nucleotides (nt) in length from an in vitro system and use of the siRNA to interfere with mRNA of a gene in a cell or organism were described in WO0175164 A2, the contents of which is entirely incorporated herein by reference.

The siRNA can also be made in vivo from a mammalian cell using a stable expression system. For example, a vector system, named pSUPER, that directs the synthesis of small interfering RNAs (siRNAs) in mammalian cells, was recently reported (Brummelkamp et al., (2002) Science 296: 550-3.), and the contents of which is incorporated herein by reference.

On the pSUPER, the H1-RNA promoter was cloned in front of the gene specific targeting sequence (19-nt sequences from the target transcript separated by a short spacer from the reverse complement of the same sequence) and five thymidines (T5) as a termination signal. The resulting transcript is predicted to fold back on itself to form a 19-base pair stem-loop structure, resembling that of C. elegans Let-7. The size of the loop (the short spacer) is preferably 9 bp. A small RNA transcript lacking a poly-adenosine tail, with a well-defined start of transcription and a termination signal consisting of five thymidines in a row (T5) was produced. Most importantly, the cleavage of the transcript at the termination site is after the second uridine yielding a transcript resembling the ends of synthetic siRNAs, that also contain two 3′ overhanging T or U nucleotides. The siRNA expressed from pSUPER is able to knock down gene expression as efficiently as the synthetic siRNA.

The present invention provides a method of treating pain in a subject in need thereof, comprising the steps of (a) introducing siRNA that targets the mRNA of the HCN gene for degradation into the cell or organism; (b) maintaining the cell or organism produced (a) under conditions under which siRNA interference of the mRNA of the HCN gene in the cell or organism occurs. The siRNA can be produced chemically via nucleotide synthesis, from an in vitro system similar to that described in WO0175164, or from an in vivo stable expression vector similar to pSUPER described herein. The siRNA can be administered similarly as that of the anti-sense nucleic acids described herein.

During treatment, the therapeutically effective dose of the composition will depend on the particular condition being treated, the severity of the condition, the individual patient parameters including age, physical condition, size and weight, the duration of the treatment, the nature of the particular agent thereof employed and the concurrent therapy (if any), the specific route of administration and like factors within the knowledge and expertise of the health practitioner. A physician or veterinarian of ordinary skill can readily determine and prescribe the effective amount of the drug required to treat or prevent the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. It is preferred generally that a maximum dose be used, that is, the highest safe dose according to sound medical judgment. It will be understood by those of ordinary skill in the art, however, that a patient may insist upon a lower dose or tolerable dose for medical, psychological or other reasons.

The daily dosage of administration of the active composition may be varied over a wide range from 0.01 to 1,000 mg per patient, per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day.

Advantageously, active compounds of the present invention may be administered in a single daily dose, or the total daily dosage can be administered in divided doses of two, three or four times daily. Furthermore, the composition can be administered topically or via transdermal routes, using, for example, transdermal patches, as are well known to those of ordinary skill in that art. Preferably, active compounds of the present invention may be administered over an extended time period to produce analgesic therapy by a transdermal controlled release-rate mechanism as described in U.S. Pat. No. 5,914,131. Dosage may also be administered intravenously, by intramuscular injection, or by injection in the vicinity of a nerve, ganglion or the spinal cord. The active compound may also be administered as a diagnostic test to evaluate whether a subject suffers from dysfunction of HCN pacemaker channels.

The active compounds of the present invention may also be administered by continuous infusion either from an external source, for example by intravenous infusion or from a source of the compound placed within the body. Internal sources include implanted reservoirs containing the compound to be infused which is continuously released, for example, by osmosis and implants which may be: (a) liquid-based such as an oily suspension of the compound to be infused for example in the form of a very sparingly water-soluble derivative such as a dodecanoate salt or a lipophilic ester; or (b) solid in the form of an implanted support, for example a synthetic resin or waxy material, for the compound to be infused. The support may be a single body containing all the compound or a series of several bodies each containing part of the compound to be delivered. The amount of active compound present in an internal source should be such that a therapeutically effective amount of the compound is delivered over a long period of time.

The active composition disclosed herein may be used alone at appropriate dosages defined by routine testing in order to obtain optimal treatment of pain while minimizing any potential toxicity. In addition, co-administration or sequential administration of other analgesics described supra may be desirable. For combination treatment with more than one active compounds, where the active compounds are in separate dosage formulations, the active compounds can be administered concurrently, or they each can be administered at separately staggered times. The dosages of administration are adjusted when several agents are combined to achieve desired effects. Dosages of these various agents may be independently optimized and combined to achieve a synergistic result wherein the pathology is reduced more than it would be if either agent were used alone.

Identification of Compounds That Are Useful for Treating Pain.

The invention further provides efficient methods of identifying compounds that are useful for pain treatment. Generally, the methods involve identifying compounds that increase or decrease: 1) the expression of an HCN pacemaker protein; 2) the open probability of an HCN pacemaker channel; or 3) the ionic conductance of a HCN pacemaker channel. Preferably, the methods further involve the step of administering the identified compound into an animal pain model to test its therapeutic effect on pain.

The compound identification methods can be in conventional laboratory format or adapted for high throughput. The term “high throughput” refers to an assay design that allows easy screening of multiple samples simultaneously, and capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid handling experiments. It is well known by those in the art that as miniaturization of plastic molds and liquid handling devices are advanced, or as improved assay devices are designed, that greater numbers of samples may be performed using the design of the present invention.

Candidate compounds encompass numerous chemical classes, although typically they are organic compounds. Preferably, they are small organic compounds, i.e., those having a molecular weight of more than 50 yet less than about 2500. Candidate compounds comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid phase or solution phase libraries: synthetic library methods requiring deconvolution; the “one-bead one-compound” library method; and synthetic library methods using affinity chromatography selection (Lam (1997) Anticancer Drug Des. 12:145). Alternatively, libraries of natural compounds in the form of bacterial, fungal, plant and animal extracts are available or readily produced. Additionally, natural and synthetically produced libraries and compounds can be readily modified through conventional chemical, physical, and biochemical means.

Further, known pharmacological agents may be subjected to directed or random chemical modifications such as acylation, alkylation, esterification, amidification, etc. to produce structural analogs of the agents. Candidate compounds can be selected randomly or can be based on existing compounds that bind to and/or modulate the function of HCN pacemaker channels. Examples include: ZD7288, ZM-227189 (Astra Zeneca), Zatebradine, DK-AH268, alinidine (Boehringer Ingelheim), ivabradine (Servier), clonidine, and lidocaine. Therefore, a source of candidate agents is libraries of molecules based on the known HCN pacemaker channel activators or inhibitors, in which the structure of the compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions. One of ordinary skill in the art in the preparation of combinatorial libraries can readily prepare such libraries based on the existing HCN pacemaker channel activators/inhibitors.

A variety of other reagents also can be included in the mixture. These include reagents such as salts, buffers, neutral proteins (e.g., albumin), detergents, etc. that may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

1. Identify Compounds That Increase or Decrease the HCN Pacemaker Protein Expression.

As used herein, “compounds that increase or decrease the HCN pacemaker protein expression” include compounds that increase or decrease HCN pacemaker gene transcription and/or translation. The invention provides a method of identifying such a compound, which comprises the steps of contacting a compound with a regulatory sequence of the HCN pacemaker gene or a cellular component that subsequently binds to the regulatory sequence; and determining the effect of the compound on the expression of a gene controlled by the regulatory sequence; wherein the regulatory sequence of the HCN pacemaker gene is either within a host cell or in a cell-free system. The term “regulatory sequence” is as defined supra.

In a preferred embodiment, the method involves a regulatory sequence of the HCN pacemaker gene within a host cell. The cell-based assay comprises the step of: (1) contacting a compound with a cell having a regulatory sequence for an HCN pacemaker gene or a cellular component that binds to the regulatory sequence for an HCN pacemaker gene; (2) measuring the effect of the compound on the expression of an HCN or reporter gene controlled by the regulatory sequence; and (3) comparing the effect of the compound with that of a reference control. The host cell can be a native HCN host cell, or a recombinant host cell. The reference control contains only the vehicle in which the testing compound is dissolved. Several assay methods can be used to measure the effect of the compound on the expression of the HCN or reporter gene inside a cell. For example, gene or protein fusions comprising the regulatory sequence for an HCN pacemaker linked to a reporter gene can be used. As used herein, “a reporter gene” refers to a gene encoding a gene product which can be measured using conventional lab techniques. Such reporter genes include but are not limited to genes encoding green fluorescent protein (GFP), β-galactosidase, luciferase, chloramphenicol acetyltransferase, β-glucuronidase, neomycin phosphotransferase, and guanine xanthine phosphoribosyl-transferase. The gene fusion is constructed such that only the transcription of the reporter gene is under control of the HCN pacemaker regulatory sequence. The protein fusion is constructed so that both the transcription and translation of the reporter gene protein are under control of the HCN pacemaker regulatory sequence. Preferably, a second gene or protein fusion comprising the same reporter gene but a different regulatory sequence (i.e., a regulatory sequence for a gene unrelated to HCN pacemaker family) can be used to increase the specificity of the assay. The effect of the compound on the expression of the reporter gene, such as GFP, can be measured by methods known to those skilled in the art. For example, the effect of the compound on expression of GFP can be measured as the effect of the compound on emissions of green fluorescence from the cell using a fluorometer. Alternatively, a cellular phenotype attributed to an HCN pacemaker channel, such as a characteristic voltage and time dependent activation profile, or a specific range of sensitivity to Cs⁺ or ZD7288, can also be used to measure the effect of the compound on the expression of the HCN pacemaker protein. In addition, the effect of the compound can be assayed by measuring the amount of HCN or reporter mRNA or protein inside the cell directly using methods described supra (i.e., Northern Blot, RT-PCR, SDS-PAGE, Western Blot, etc.).

Note that the cell-based method described supra not only identifies compounds that regulate HCN expression directly via binding to the regulatory sequence of an HCN gene, but also identifies compounds that regulate HCN expression indirectly via binding to other cellular components whose activities influence the HCN expression. For example, compounds that modulate the activity of a transcriptional activator or inhibitor for HCN genes can be identified using the method described herein.

In another embodiment, the method involves a regulatory sequence of the HCN pacemaker gene in a cell-free assay system. The cell-free assay comprises the step of: (1) contacting a compound to the regulatory sequence for an HCN pacemaker gene or a cellular component that binds to the regulatory sequence for an HCN pacemaker gene in a cell-free assay system; (2) measuring the effect of the compound on the expression of the HCN or reporter gene controlled by the regulatory sequence; and (3) comparing the effect of the compound with that of a reference control. The reference control contains only the vehicle in which the testing compound is dissolved. Examples of the cell-free assay system include the in vitro translation and/or transcription system, which are known to those skilled in the art. For example, the full length HCN pacemaker cDNA, including the regulatory sequence, can be cloned into a plasmid. Then, using this construct as the template, HCN pacemaker protein can be produced in an in vitro transcription and translation system. Alternatively, synthetic HCN pacemaker mRNA or mRNA isolated from HCN pacemaker protein producing cells can be efficiently translated in various cell-free systems, including but not limited to wheat germ extracts and reticulocyte extracts. The effect of the compound on the expression of the HCN or reporter genes controlled by the regulatory sequence can be monitored by direct measurement of the quantity of HCN or reporter mRNA or protein using methods described supra.

2. Methods of Identifying an Inhibitor or Activator of an HCN Pacemaker Channel.

“Inhibitors” or “blockers”, “activators” or “openers,” and “modulators” of HCN pacemaker channels refer to inhibitory or activating molecules identified using in vitro and in vivo assays for HCN channel function. In particular, “inhibitors” or “blockers”, refer to compounds that decrease, block, prevent, delay activation, inactivate, desensitize or down regulate channel activity, or speed or enhance deactivation of the channel. “Activators” or “openers” are compounds that increase, open, activate, facilitate, enhance activation, sensitize or upregulate channel activity, or delay or slow inactivation. “Modulators” include both the “inhibitors” and “activators”.

The invention further provides a method of identifying an inhibitor or an activator of an HCN pacemaker channel. The method comprises the steps of contacting a test compound with an HCN pacemaker subunit; and determining the effect of the compound on the function of an HCN pacemaker channel.

The amount of time necessary for cellular contact with the compound is empirically determined, for example, by running a time course with an HCN pacemaker modulator, such as ZD7288, and measuring cellular changes as a function of time.

The term “function” as used herein refers to the expression of an HCN pacemaker characteristic activity. For example, but not by way of limitation, the function of an HCN channel may be measured by the Iₕ current conducted by the channel, the voltage- and time-dependent activation of the channel, and the sensitivity of channel to Cs⁺ and ZD7288.

A variety of assay methods can be used to determine the effect of the compound on the function of an HCN pacemaker channel. Some of the screening methods are illustrated herein in examples 13-15 without limiting the scope of the invention. In one preferred embodiment, compounds that increase or decrease the Iₕ current density can be identified by contacting a test compound with an HCN channel, and measuring Iₕ current with patch-clamp techniques or voltage-clamp techniques under different conditions, or by measuring ion flux with radioisotope or non-radioisotope flux assays, or fluorescence assays using voltage-sensitive dyes (See, e.g., Vestergarrd-Bogind et al., (1988), J. Membrane Biol., 88: 67-75; Daniel et al., (1991), J. Pharmacol. Meth., 25:185-193; Holevinsky et al., (1994), J. Membrane Biology, 137: 59-70). Preferably, recombinant host cells that express recombinant HCN subunit, cell membranes prepared from the recombinant host cells, or substantially purified HCN protein incorporated into lipid bilayers are used for the assay. As used herein “recombinant HCN subunit” refers to an HCN subunit produced by recombinant DNA techniques; i.e., produced from cells transformed by an exogenous DNA construct encoding the HCN subunit. Alternatively, native host cells expressing endogeneous HCN channels, such as DRG cells, or membrane proteins from the native host cell, can also be used for the assay. Convenient reagents for such assay methods are known in the art. Exemplary assays are described herein.

To examine the extent of inhibition, samples or assays comprising an HCN channel are treated with a potential activator or inhibitor compound and are compared to control samples without the test compound. Control samples (untreated with test compounds) are assigned a relative HCN activity value of 100%. Inhibition of channels comprising an HCN subunit is achieved when the HCN activity value relative to the control is about 75%, preferably 50%, more preferably 25-0%. Activation of channels comprising an HCN subunit is achieved when the HCN activity value relative to the control is 110%, more preferably 150%, most preferably at least 200-500% higher or 1000% or higher.

The measurement means of the method of the present invention can be further defined by comparing two cells, one containing an HCN channel subunit and a second cell originating from the same clone but lacking the HCN channel subunit. After both cells are contacted with the same test compound, differences in HCN activities between the two cells are compared. This technique is also useful in establishing the background noise of these assays. One of ordinary skill in the art will appreciate that these control mechanisms also allow easy selection of cellular changes that are responsive to modulation of functional HCN channels.

The term “cell” refers to at least one cell, but includes a plurality of cells appropriate for the sensitivity of the detection method. Cells suitable for the present invention may be bacterial, yeast, or eukaryotic.

In another preferred embodiment, binding assays can be used to identify to a compound that binds to an HCN subunit, and potentially is capable of inhibiting or activating the function of an HCN channel comprising such an HCN subunit. One exemplary method comprising the steps of: (a) incubating an HCN subunit with a labeled ligand for an HCN subunit, such as a radioactive ZD7288, and a test compound, and where the contact is for sufficient time to allow the labeled ligand to reach equilibrium binding to the HCN subunit; (b) separating the HCN subunit from unbound labeled ligand; and (c) identifying a compound that inhibits ligand binding to the subunit by a reduction in the amount of labeled ligand binding to the HCN subunit. Preferably, an HCN host cell (recombinant or native) that expresses the HCN subunit can be used for the binding assay. More preferably, membranes prepared from the HCN host cell can be used for the binding assay. Further preferably, a substantially purified HCN subunit protein can be used for the binding assay.

As used herein, the term “substantially purified” means that the protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals that are involved in the synthesis of the protein. Accordingly such preparations of the protein have less than about 30%, 20%, 10%, 5% (by dry weight) of chemical precursors or compounds other than the polypeptide of interest.

Separation of the HCN subunit from unbound labeled ligand can be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximize signal to noise ratios, primarily to minimize background binding, as well as for ease of separation and cost.

Separation may be effected for example, by removing a bead or dipstick from a reservoir, emptying or diluting a reservoir such as a microtiter plate well, or rinsing a bead, particle, chromatographic column or filter with a wash solution or solvent. The separation step preferably includes multiple rinses or washes. For example, when the solid substrate is a microtiter plate, the wells may be washed several times with a washing solution, that typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

A wide variety of labels can be used to label the HCN ligand, such as those that provide direct detection (e.g., radioactivity, luminescence, optical or electron density, etc.), or indirect detection (e.g., epitope tag such as the FLAG epitope, enzyme tag such as horseradish peroxidase, etc.).

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Yet another assay for identifying compound that increases or decreases ion flux through an HCN pacemaker channel involves “virtual genetics”. The method of which is described in WO 98/11139 and is fully incorporated herein.

The following examples illustrate the present invention without, however, limiting the same thereto. The contents of all references, patents and published patent applications cited throughout this application are hereby incorporated by reference.

### EXAMPLE 1

### Cloning of Human HCN1 and HCN3 cDNA

1. Cloning of Human HCN1 cDNA.

We used the partial rat HCN1 coding region sequence (Genbank accession ID AF155163) to query the human genome draft sequence to identify putative human HCN1 translation start and stop sites. These were identified within GenBank htgs contigs # AC013384 and AC026621. Two primers, SEQ ID NO: 1, 5′ ACG TAA GCT TGC CAC CAT GGA AGG AGG CGG CAA GCC CAA C 3′ and SEQ ID NO: 2, 5′ ACG TAG GCG GCC GCT CAT AAA TTT GAA GCA AAT CGT GGC T 3′, were used to PCR amplify the human HCN1 coding region using human spinal cord cDNA as template. A 2.7 kb PCR fragment was cloned into pcDNA 3.1/Zeo and the complete human HCN1 cDNA was sequenced. The nucleotide sequence of the complete human HCN1 is depicted in SEQ ID NO: 3, and the deduced amino acid sequence of human HCN1 protein is shown in SEQ ID NO: 4.

2. Cloning of Human HCN3 cDNA

We used the rat HCN3 cDNA sequence (#AF247452) to query the Genbank DNA database to identify putative human HCN3 cDNA. A partial cDNA (AB040968) encoding KIAA1535 protein with high homology to the 3′ end of rat HCN3 was identified. Two primers, SEQ ID NO: 5, 5′CCTCCTCCACCACGATGCCCGTTCGGAAGTGAG 3′ (designed from AB040968) and SEQ ID NO: 6, 5′CCATCCTAATACGACTCACTATAGGGC 3′ (an adaptor) were used to PCR amplify the 5′ end of human HCN3 using human brain Marathon-ready cDNA (Clontech) as template. The resulting amplicon was sequenced to obtain the 5′ end sequence of human HCN3. Two primers, SEQ ID NO: 7: 5′ATCAAAGCTTGCCACCATGGAGGCAGAGCAGCGGCCGGCGG 3′ and SEQ ID NO: 8: 5′ACGTACGCGGCCGCTTACATGTTGGCAGAAAGCTGGAGAC 3′ were then used to amplify the complete human HCN3 cDNA. The resulting 2.3 kb PCR fragment was cloned into a mammalian expression vector, pcDNA 3.1/Zeo (Invitrogen), and an oocyte expression vector, pGEMHE, and the complete human HCN3 cDNA was sequenced. The nucleotide sequence of the complete human HCN3 is depicted in SEQ ID NO: 9, and the deduced amino acid sequence of human HCN3 protein is shown in SEQ ID NO: 10.

The 2325 base pairs nucleotide sequence of human HCN3 revealed a single large open reading frame encoding a polypeptide of 774 amino acids. The first in-frame methionine was designated as the initiation codon for an open reading frame that predicts a human hyperpolarization-activated cation-nonselective cyclic-nucleotide modulated protein with an estimated molecular mass (Mᵣ) of about 86 kDa.

### EXAMPLE 2

### Characterization of Functional Protein Encoded by HCN1 in Xenopus oocytes

Xenopus laevis oocytes were prepared and injected using standard methods previously described and known in the art [Fraser et al. (1993). Electrophysiology: a practical approach. D. I. Wallis, IRL Press at Oxford University Press, Oxford: 65-86]. Ovarian lobes from adult female Xenopus laevis (Nasco, Fort Atkinson, Wis.) were teased apart, rinsed several times in nominally Ca-free saline containing: 82.5 mM NaCl, 2.5 mM KCl, 1 mM MgCl₂, 5 mM HEPES, adjusted to pH 7.0 with NaOH (OR-2), and gently shaken in OR-2 containing 0.2% collagenase Type 1 (ICN Biomedicals, Aurora, Ohio) for 2-5 hours. When approximately 50% of the follicular layers were removed, Stage V and VI oocytes were selected and rinsed in media consisting of 75% OR-2 and 25% ND-96. The ND-96 contained: 100 mM NaCl, 2 mM KCl, 1 mM MgCl₂, 1.8 mM CaCl₂, 5 mM HEPES, 2.5 mM Na pyruvate, gentamicin (50 ug/ml), adjusted to pH 7.0 with NaOH. The extracellular Ca⁺² was gradually increased and the cells were maintained in ND-96 for 2-24 hours before injection. For in vitro transcription, pGEM HE (Liman et al., (1992) Neuron 9: 861-71) containing human HCN1 was linearized with NheI and transcribed with T7 RNA polymerase (Promega) in the presence of the cap analog m7G(5′)ppp(5′)G. The synthesized cRNA was precipitated with ammonium acetate and isopropanol, and resuspended in 50 μl nuclease-free water. cRNA was quantified using formaldehyde gels (1% agarose, 1×MOPS , 3% formaldehyde) against 1,2 and 5 μl RNA markers (Gibco BRL, 0.24-9.5 Kb).

Oocytes were injected with 50 nl of human HCN1 RNA (1-10 ng). Control oocytes were injected with 50 nl of water. Oocytes were incubated for 2 days in ND-96 before analysis for expression of human HCN1. Incubations and collagenase digestion were carried out at room temperature. Injected oocytes were maintained in 48 well cell culture clusters (Costar; Cambridge, Mass.) at 18° C. Whole cell voltage-activated currents were measured 2 days after injection with a conventional two-electrode voltage clamp (GeneClamp500, Axon Instruments, Foster City, Calif.) using standard methods previously described and known in the art (Dascal et al., (1987) Pflugers Arch 409: 512-20). The microelectrodes, which had resistances of ˜1 MΩ, were filled with 3 M KCl. Cells were continuously perfused with ND96 at ˜10 ml/min at room temperature. Membrane voltage was clamped at −30 mV unless indicated.

Oocytes were challenged with a series of hyperpolarizing voltage steps from a holding potential of −30 mV. Voltage steps (800 msec duration) to −40 through −180 mV in 20 mV intervals were applied to oocytes at a sampling rate of 0.1 Hz. Hyperpolarizing voltage steps activated an inward current having a threshold of about −60 mV. Control-injected oocytes revealed no inward currents at potentials below about −120 mV, however, an endogenous inward current was activated in both HCN- and control-injected oocytes having a threshold near −120 mV. The inward currents observed in HCN1-injected oocytes at −60, −80 and −100 mV were significantly larger than the currents observed in control oocytes (p<0.05) (FIG. 1). Cs⁺ (3 mM added to ND-96 as CsCl) reversibly blocked the low threshold inward currents in HCN1-injected oocytes (n=3) however, Cs⁺ had no effect on the endogenous currents. Cs⁺ blocked HCN1 currents by 94, 92 and 90% when Iₕ was evoked by stepping to −80, −100 and −120 mV, respectively. Thus, HCN1-injected oocytes expressed a hyperpolarization-activated inward current that was sensitive to external Cs⁺ and was consistent with an Iₕ current.

### EXAMPLE 3

### Functional Characterization of Human HCN3 Subunit in a Mammalian Expression System by Whole Cell Patch Clamp

1. Expression of the Functional Cloned Human HCN3 in HEK293 Cells

Stable transfection: Semi-adherent, confluent wild-type Human Embryonic Kidney (HEK) cells were incubated in the presence of 0.25% trypsin/1 mM EDTA-4Na until dislodged, diluted in HEK medium [DMEM (Gibco, Grand Island, N.Y.) +10% fetal bovine serum (FBS)+1:200 pen/strep] and plated in 10 cm dishes at a sufficient density to ensure 75% cell confluence following overnight incubation at 37° C. Transfections were performed using Superfect (Qiagen, Chatsworth, Calif.) according to the manufacturer's protocol. A transfection mixture comprising 10 micrograms of hHCN3-pCDNA3.1 Zeo plasmid DNA diluted in serum-free DMEM medium (Gibco) (a final volume of 0.3 ml) and 0.06 ml of Superfect reagent (Qiagen) was vortexed for 10 seconds and incubated at room temperature for 10 minutes to facilitate liposome/DNA complex formation. Then, adherent cells washed once in serum-free DMEM medium were added to the transfection mixture (supplemented with 3 mls of DMEM medium). After 2 hours incubation at 37° C., the cells in the transfection mixture were grown overnight at 37° C. in fresh HEK medium. Forty-eight hours following transfection, the cells were passaged into 15 cm culture dishes and maintained under zeocin (400 μg/ml) selection (Invitrogen, Carlsbad, Calif.), and individual cell colonies were selected and tested electrophysiologically for the expression of HCN currents.

Confirmation of hHCN3 plasmid-containing cell clones by PCR: hHCN3 transfected cell lines revealing hyperpolarization-activated currents and untransfected HEK 293 cells were grown in 10 cm culture dishes until 80% confluent. Total RNA was isolated from the cells with Trizol reagent (Gibco) according to the manufacturer's protocol. Following spectrophotometric quantification, 1 microgram of total RNA, isolated from both untransfected and potential hHCN3-expressing HEK293 cells, was reverse-transcribed into cDNA with Superscript II reverse transcriptase (Gibco) according to the manufacturer's protocol. Synthesized cDNAs were diluted 1:5 in nuclease-free H₂O supplemented with poly-inosine to a final concentration of 10 nanograms per ml, heated at 70° C. for five minutes and placed on ice for an additional 2 minutes. Diluted cDNA was used as template for LightCycler® PCR (Roche, Indianapolis, Ind.) in accordance with user-defined protocols. Primer sequences used in LightCycler PCR were selected to permit positive identification of hHCN3 plasmid-derived transcripts as well as to reveal possible endogenous HEK 293 hHCN3 expression. These primer sequences included, SEQ ID NO: 11, 5′ AGCTTCGTCACTGCAGTTCTCACC 3′(hHCN3 gene-specific sense oligo), SEQ ID NO: 12, 5′ AGCCATGTCTCTGTCATGTTGCACC 3′(hHCN3 gene-specific antisense oligo), and SEQ ID NO: 13, 5′ AGTGGCACCTTCCAGGGTCAA 3′ (pcDNA3.1 Zeo plasmid-specific antisense oligo).

PCR products were fractionated by ethidium bromide agarose gel electrophoresis and visualized under ultraviolet light. Amplicons of the predicted molecular weight were subcloned into the pCR4-TOPO TA cloning vector according to the manufacturer's protocol and sequenced to confirm sequence identity. Indeed, hHCN3 gene specific sequences were successfully amplified and identified from stably transfected cells but not control cell lines.

2. Characterization of Human Hyperpolarization-activated Non-selective Cation Channel HCN3 in the Human HEK293 Cell Line.

Patch clamp: The whole cell patch clamp technique (Hamill et al., (1981) Pflugers Arch 391: 85-100) was used to record voltage-activated currents from HEK293 stably expressing human HCN3 obtained above. The transfected cells were maintained for more than 1 day on 12 mm coverslips. Cells were visualized using a Nikon Diaphot 300 with DIC Nomarski optics. Cells were continuously perfused in physiological solution (˜0.5 ml/min) unless otherwise indicated. The standard physiological solution used (1Ca Tyrode's (“Tyrode's”) contained: 130 mM NaCl, 4 mM KCl, 1 mM CaCl₂, 1.2 mM MgCl₂, and 10 mM hemi-Na-HEPES (pH 7.3, 295-300 mOsm as measured using a Wescor 5500 vapor-pressure (Wescor, Inc., Logan, Utah)). Recording electrodes were fabricated from borosilicate capillary tubing (R6; Garner Glass, Claremont, Calif.), the tips were coated with dental periphery wax (Miles Laboratories, South Bend, Ind.), and had resistances of 1-2 MΩ when containing the following intracellular solution: 100 mM K-gluconate, 25 mM KCl, 0.483 mM CaCl₂, 3 mM MgCl₂, 10 mM hemi-Na-HEPES and 1 mM K₄-BAPTA (100 nM free Ca⁺²); pH 7.4, with dextrose added to achieve 290 mOsm). Liquid junction potentials were −14 mV using standard pipette and bath solutions, as determined both empirically and using the computer program JPCalc(Barry, (1994) J Neurosci Methods 51: 107-16). All voltages shown were corrected for liquid junction potential. Current and voltage signals were detected and filtered at 2 kHz with an Axopatch 1D patch-clamp amplifier (Axon Instruments, Foster City, Calif.), digitally recorded with a DigiData 1200B laboratory interface (Axon Instruments), and PC compatible computer system and stored on magnetic disk for off-line analysis. Data acquisition and analysis were performed with PClamp software.

Parameter determination: The total membrane capacitance (Cₘ) was used to normalize currents to cell size. Cₘ was determined as the difference between the maximum current after a 30 mV hyperpolarizing voltage ramp from −64 mV generated at a rate of 10 mV/ms and the steady state current at the final potential (−94 mV)(Dubin et al., (1999) J Neurosci 19: 1371-81; Dubin et al., (1999) J Biol Chem 274: 30799-810). Since Iₕ develops slowly, the current reached steady state prior to the onset of the hyperpolarization-induced currents.

Whole cell hyperpolarization-induced currents were determined as the difference between the initial baseline current at the end of the capacitative transient and the maximum inward current at the end of a 1-3 sec voltage step. The values determined were not significantly different from the current that was blocked by 3 mM CsCl, which completely blocks Iₕ, in the same cells. Membrane potential was held at −64 mV between voltage pulses and the current required to clamp the cells at −64 mV was continuously monitored. The voltage protocol for Iₕ activation included a step to −54 mV prior to the family of voltage steps to activate Iₕ.

The kinetics of activation were determined using Chebyshev with 4-pt smoothing filter fit in the Pclamp CLAMPFIT suite of programs (Axon Instruments). The currents could be best described by a 2 exponential fit.

Apparent reversal potentials (Vᵣₑᵥ; the voltage at which there was no current) of hyperpolarization activated conductance changes were determined using either a voltage-ramp protocol (Dubin et al., (1999) J Neurosci 19: 1371-81; Dubin et al., (1999) J Biol Chem 274: 30799-810) or tail current analysis. Membrane potential was held at −64 mV between voltage pulses. Every 2 sec the membrane potential was stepped to −164 mV for 450 msec to nearly fully activate Iₕ followed by a voltage ramp from −164 mV to +36 mV at a rate of 0.5 mV/msec. The resulting whole cell voltage step- and ramp-induced currents were recorded without on-line leak subtraction in the presence and absence of 3 mM CsCl. Vᵣₑᵥ was the voltage at which the Cs-sensitive current was 0 (the voltage at which the current voltage relationships in the presence and absence of Cs crossed each other) or the voltage at which the ramp-induced currents merged. Tail currents were measured by activating Iₕ and then stepping to −84 to −14 mV in increments of 10 mV. In tail current analysis, Vᵣₑᵥ was the voltage at which the deactivating current reversed sign. Controls were done in the presence of extracellular Cs⁺ to block Iₕ to show that the currents elicited by voltage steps to −30 and more positive were not confounded by the activation of endogenous outward currents. At this low Cs⁺ concentration there was little or no effect on endogenous outward K⁺ currents and Cs⁺ strongly blocked the currents elicited at membrane potentials more negative than about −40 mV.

Results: Currents with the activation, kinetic and pharmacological characteristics of Iₕ were observed in human HCN3-transfected cell lines. These currents were not observed in control lines. Hyperpolarizing voltage pulses activated a slowly developing inward current in HCN3/HEK cells (FIG. 2). The threshold of activation of the currents was −87+/−2 mV (n=10). The threshold values were similar for 5 independent cell lines tested. The hyperpolarization-induced inward currents (“Iₕ”) were completely blocked by 3 mM CsCl. The effect of Cs⁺ was rapidly reversible. At very negative voltages, a noisy current developed that was not sensitive to Cs⁺. The specific antagonist ZD7288 (Tocris Cookson Inc., Ballwin, Mo.), (50 μM) blocked the currents by 98+/−2% (n=3) The slow development of block was consistent with previous reports and is likely due to ZD7288 binding to the intracellular pore vestibule (Shin et al., (2001) Biophysical Journal 80: 337a); the effect of ZD7288 did not reverse during a washout period of at least 15 min. When new cells were selected for recording that had been previously exposed to bath application of ZD7288 (50 μM) and subsequently washed, it was found that 3 of 3 cells showed no detectable Iₕ, whereas previously 6 of 6 cells tested expressed Iₕ. Thus, HCN3 mediated currents were sensitive to both Cs⁺ and ZD7288. Hyperpolarization activated currents in HCN3/HEK cells had a reversal potential similar to that reported in the literature and consistent with mediation by both K⁺ and Na⁺. By tail current analysis, Vrev was −44+−4 mV (n=4). Similar results were obtained from Cs⁺ sensitive currents measured using a voltage ramp protocol (−41+/−5 mV; n=3).

### EXAMPLE 4

### Specific Blockade of HCN Channels Suppressed Spontaneous Firing of Injured Primary Afferents in an Animal Neuropathic Pain Model

Male Sprague Dawley rats (Harlan, Indianapolis, Ind.), weighing 120-150 g, were used for the experiments. The animals were housed in groups of two in plastic cages, with corn chip bedding under a 12/12 hour reversed light-dark cycle (light cycle was 9:00 PM to 9:00 AM), with a constant ambient temperature and free access to food and water. Surgery for the spinal nerve ligation was performed as previously described (Kim et al., (1992) Pain 50: 355-363) with the modification for electrophysiological study of ligation of L4 and L5 instead of L5 and L6; electrophysiology methods were as previously published, and as summarized below (Lee et al., (1999) J Neurophysiol 81: 2226-33).

Single unit recordings were made from the L4 or L5 dorsal root filaments at a time between postoperative day 7 and 23. Under isoflurane anesthesia, the L4 and L5 DRGs, along with dorsal roots and spinal nerves, were removed. The DRG was placed in an in vitro recording chamber with separate compartments for the DRG and the spinal nerve versus the dorsal root. The DRG/spinal nerve compartment was perfused with oxygenated (95% O₂ and 5% CO₂) artificial cerebrospinal fluid (ACSF; composition in mM: NaCl 130, KCl 3.5, NaH₂PO₄ 1.25, NaHCO₃ 24, Dextrose 10, MgCl₂ 1.2, CaCl₂ 1.2, pH=7.3) at a rate of 4-5 ml/min. The dorsal root compartment was filled with mineral oil. The temperature was kept at 34±1° C. through a temperature controlled water bath. Ectopic discharges were recorded from the teased dorsal root fascicles and the spinal nerve was stimulated using a tungsten bipolar (1 mm gap) electrode. Fiber types were classified according to their conduction velocity: >14 m/sec for Aβ, 2 -14 m/sec for Aδ, and <2 m/sec for C fibers. Fine filaments were dissected until single spontaneous units (>1 Hz) could be isolated on the basis of the amplitude and waveform. Neural activity was amplified with an AC-coupled amplifier (WPI, ISO-80A) and the output then fed to a window discriminator (WPI, N-750). The output of the window discriminator was used to construct peristimulus time histograms (PSTHs) by a data acquisition system (CED-1401, spike 2). Unit recordings were made from teased dorsal root fibers. Once a spontaneously active unit was found, baseline activity was recorded for at least 10 minutes. If the activity was not stable (continuous increase or decrease) during this baseline measurement, the baseline period was extended until a full 10 minutes of stable activity was recorded or the unit was discarded and another fiber was teased. During the baseline recording, the action potential was sampled and stored into a digital oscilloscope (Tektronix,) for comparison to electrically evoked activity at the end of recording for determination of conduction velocity. Once a stable baseline was obtained, ZD7288 dissolved in ACSF was added to the perfusate for 5 minutes. For control experiments, ACSF was applied for 5 minutes through the same route as with ZD7288 application. After beginning the drug application, unit activity was monitored for 30 minutes. Conduction velocity (CV) was measured at the end of experiment because electrical stimulation often changed the firing pattern of the units. At the end of the experiment, ectopic discharge was usually still present, although the firing rate was decreased. However, some units exposed to the highest dose of ZD7288 (100 uM) were still silent at the end of the 30 min observation period. If the unit was totally lost, CV was measured by referring to the digitally stored action potential.

The numbers of spikes during 5 minute bins were calculated over a 40 minute period (10 minutes baseline and remaining 30 minutes) (FIG. 3). Each number was transformed to percentage of change from the firing frequency during the first 10 minutes (FIG. 4). Data were expressed as mean±standard error of the mean (S.E.M.). Statistical analyses were performed by one-way ANOVA followed by Dunnett's multiple comparisons in each time point.

### EXAMPLE 5

### Specific Pharmacological Blockade of HCN Channels Selectively Suppresses the Neuropathic Pain Behavior Seen in the SNL (Chung) Model

ZD7288 (BoSmith et al., (1993) Br J Pharmacol 110: 343-9) has been reported to suppress Iₕ in peripheral nerves (Takigawa et al., (1998) Neuroscience 82: 631-4) and DRG neurons(Cardenas et al., (1999) J Physiol (Lond) 518: 507-23; Yagi et al., (1998) J Neurophysiol 80: 1094-104). Suppression of repetitive action potentials in in vitro DRG preparations with attached sciatic nerve fragments from previously sciatic nerve ligated rats with ZD7288 has also recently been reported (Yagi et al., (2000) Proceedings of the 9th World Congress on Pain 16: 109-117).

Preparation of SNL (spinal nerve ligation) model: All studies were conducted in keeping with the guidelines of the Institutional Animal Care Committees of the University of California, San Diego and RWJPRI. Male Harlan Sprague-Dawley rats, 100-150 g, were housed in cages with solid bottoms and sawdust bedding, with a 12/12 h reversed light cycle (lights on 2100-900), and allowed free access to food pellets and water. Animals were housed in groups of 2 after surgical interventions. A surgical neuropathy was created as follows, to create a model commonly referred to as the SNL, or spinal nerve ligation model, also commonly referred to as the Chung model. Under isoflurane/oxygen anesthesia, a dorsal midline incision was made from approximately L3-S2. Using a mixture of sharp and blunt dissection, the left L6/S1 posterior interarticular process was exposed and resected to permit adequate visualization of the L6 transverse process, which was gently removed. Careful teasing of the underlying fascia exposed the left L4 and L5 spinal nerves distal to their emergence from the intervertebral foramina. The nerves were gently separated, and the L5 and in some cases either the L4 (for in vitro electrophysiology recordings) or the L6 nerve firmly ligated with 6-0 silk suture material. The wound was then inspected for hemostasis and closed in two layers. Animals with thresholds greater than 4 g were considered unsuccessful preparations(Chaplan et al., (1994) Journal of Neuroscience Methods 53: 55-63). Note that in some versions of this procedure, both L5 and L6 are ligated; however, it has been shown that behavioral outcomes with L5 section or ligation alone are comparable to ligation of both L5 and L6 (Kinnman et al., (1995) Neuroscience 64: 751-67).

Behavioral assessment: Behavioral signs of allodynia were documented as follows. Briefly, rats were transferred to a testing cage with a wire mesh bottom and allowed to acclimate for 10-15 minutes. Von Frey filaments (Stoelting, Wood Dale Ill.) were used to determine the 50% mechanical threshold for foot withdrawal, using the up-down method of (Dixon, (1980) Annual Review of Pharmacological Toxicology 20: 441-462)Dixon as adapted by Chaplan et al (Chaplan et al., (1994) Journal of Neuroscience Methods 53: 55-63). A series of calibrated filaments, designated 3.61,3.84, 4.08, 4.17, 4.31, 4.56, 4.74, 4.93, 5.18 by the manufacturer (Stoelting, Wood Dale, Ill.) starting with one that possessed a buckling weight of approximately 2.5 g, was applied in sequence to the plantar surface of the left hindpaw with a pressure that caused the filament to buckle. Lifting of the paw was recorded as a positive response and the next lightest filament chosen for the next measurement. Absence of a response after 5 seconds prompted use of the next filament of increasing weight. This paradigm was continued until four measurements had been made after an initial change in the behavior or until five consecutive negative (given the score of 15 g) or positive (score of 0.35 g) scores had occurred. The resulting sequence of positive and negative scores was used to interpolate the 50% response threshold as previously described(Chaplan et al., (1994) Journal of Neuroscience Methods 53: 55-63).

Drug administration: After baseline documentation of allodynia, ZD7288 diluted in physiologic saline was administered to groups of rats at 10, 3 and 1 mg/kg, i.p. Paw thresholds were tested at 0.5, 1, 2, 4 and 24 hours after the administration. To compare dose and drug effects, raw paw thresholds were normalized as percent of maximum possible drug effect (% MPE) using the following formula: % MPE=[post-drug threshold(g)−predrug allodynia baseline threshold (g)]/[Pre-ligation baseline threshold (g)]−predrug allodynia baseline threshold(g)]×100. Pre-drug maximum allodynia (baseline) thresholds were assumed to reflect 0% drug effect (no suppression of allodynia) and pre-ligation threshold values were designated as 100% effect, i.e., a drug effect causing return of the paw threshold to a normal, pre-ligation baseline was taken to represent complete suppression of allodynia.

Results: ZD7288 suppressed allodynic responses in a dose-dependent manner, with an efficacy of 75.7+/−15.4% and an ED50 of approximately 3 mg/kg. No untoward behavioral effects were seen; rats had normal motor function. See FIG. 5.

### EXAMPLE 6

### The Antiallodynic Effects of ZD7288 Are Not Due to Numbness or Motor Deficits and ZD7288 Is Not a General Analgesic

Behavioral assessment: To evaluate drug effects on an acute thermally induced pain state, the hot plate test was performed, by placing the rat on a surface of 55° C. and observing the latency to paw lick, in seconds. A cutoff of 20 seconds of thermal surface exposure was employed to prevent tissue damage. Drug administration: Normal rats were administered ZD7288, 10 mg/kg, or an equivalent volume of saline, i.p. at time 0. Behavioral assessments were performed at 45, 60 and 75 minutes after drug administration.

Results: No statistically significant difference was seen between treatment with ZD7288 and saline at 45 or 60 min; a statistically significant, but very minor, difference was seen at 75 min (approximately 15%). Thus, specific blockade of HCN channels does not yield analgesia of a clinically relevant magnitude against acute thermal stimuli; the antiallodynic effects in the SNL model are selective. In addition, these results demonstrate that ZD7288 does not impair the ability of rats to respond to perceived noxious stimuli; thus, the effect of ZD7288 on allodynia thresholds is not due to inhibition of motor responses or cognitive depression. See FIG. 6.

### EXAMPLE 7

### CFA-induced Tactile Allodynia Was Blocked by Specific Pharmacological Blockade of HCN Channels

A total of 25 male Sprague Dawley rats (Harlan, Indianapolis, Ind.), weighing 230-280 g, were used for the experiments. The animals were housed in groups of two in plastic cages, with corn chip bedding under a 12/12 hour reversed light-dark cycle (light cycle was 9:00 PM to 9:00 AM), with a constant ambient temperature and free access to food and water. Following baseline testing of mechanical withdrawal thresholds (see below), complete Freund's adjuvant (CFA; 50%/10 ul, dissolved in saline, Sigma, St. Louis, Mo., U.S.A.) was injected (s.c.) in the plantar surface of the left hind paw under gaseous anesthesia with isoflurane in O₂. Twenty-four hours later, mechanical sensitivity was again measured by determining the median 50% foot withdrawal threshold for von Frey filaments using the up-down method (Chaplan et al., 1994). The rats were placed under a plastic cover (9×9×20 cm) on a metal mesh floor. The area tested was the middle glabrous area between the footpads of the plantar surface of the CFA-injected hind paw. The plantar area was touched with a series of 12 von Frey hairs with approximately logarithmic incremental bending forces (von Frey values: 3.61, 3.80, 4.00, 4.20, 4.61, 4.80, 5.00, 5.20, 5.40, 5.60, 5.80; equivalent to: 0.41, 0.63, 1, 1.58, 2.51, 4.07, 6.31, 10, 15.8, 25.1, 39.8 and 63.1 g). The von Frey hair was presented perpendicular to the plantar surface with sufficient force to cause slight bending against the plantar surface, and held for approximately 2-3 seconds. Abrupt withdrawal of the foot (paw flinching) was recorded as a response. Immediately after the baseline measurement, vehicle (saline) or one of ZD7288 (10 mg/kg), ibuprofen (30 mg/kg), morphine (3 mg/kg) was administered intraperitoneally. The von Frey test was repeated every 30 min. or 1 hr up to 4 hrs after the compound administration.

### EXAMPLE 8

### Spontaneous Pain in the Rat Mild Thermal Injury Model Was Blocked by Specific Pharmacological Blockade of HCN Channels

A standardized first-degree burn injury was induced in rats (Lofgren et al., (1998) Neuropeptides 32: 173-177). Under deep volatile anesthesia with a mixture of isoflurane (4%) in O₂ an 84 g weight was placed on the dorsum of the animal's left hind foot while the plantar surface was contacted atop a moistened hotplate (56° C.) for 20 seconds. Ten minutes after this burn injury, vehicle (saline), morphine (3 mg/kg) or ZD7288 (10 mg/kg) was injected intraperitoneally.

Spontaneous pain was assessed 0.5 and 1 hour after the compound or vehicle injection in each group. To assess spontaneous pain, the animal was placed under a transparent plastic cover on a metal mesh floor. Ten minutes were allowed for acclimatization. Following acclimatization, the cumulative amount of time during which the foot was lifted off the floor, or held in a guarded posture, was measured during specified 10-minute intervals as above. Foot lifts associated with locomotion or grooming were not counted. At 3 mg/kg, efficacy of morphine suppression of spontaneous flinching and guarding was about 89.6+/−2.1% (average of 30 min and 60 min time points: mean+/−SEM; P<0.0001 vs. saline, 1 way ANOVA with Fisher's PLSD). Similarly, efficacy of ZD7288 was about 89.1+/−15.7% (P<0.0001 vs. saline, 1 way ANOVA with Fisher's PLSD) (FIG. 8).

### EXAMPLE 9

### Alterations in Levels of HCN Message RNAs and Proteins in the DRG of the SNL Model of Neuropathic Pain

Methods: Rats were prepared with SNL (L5/6) or sham ligation as detailed above. Behavioral testing was carried out as above to document the presence of allodynia in neuropathic rats, and absence in sham rats.

RNA quantification: One week after surgery, total RNA was extracted from left L5/L6 DRGs for each rat (RNEasy, Qiagen). Conventional first strand cDNA synthesis was performed on 1/10^{th} of the yield using Superscript II (Life Technologies); 1/16^{th} of the resulting preparation was used as template per PCR reaction. Samples were simultaneously analyzed using an iCycler® (BioRad, Inc.), with Qiagen Taq Master Mix (Qiagen, Valencia, Calif.) with 1:1000 Sybr Green (Molecular Probes, Inc.) per reaction. Forward and reverse primers (Genset, La Jolla, Calif.) were as follows: HCN1: bases 308-329 and 548-570 of Genbank# AF247450 (NM_{—}053375); HCN2: 332-349 and 464-492 of Genbank# AF247451; HCN3: 140-157 and 318-337 of Genbank# AF247452 (NM_{—}053685); and HCN4: 589-610 and 777-805 of Genbank# AF247453. These PCR amplicons spanned large introns to preclude genomic DNA amplification. In addition, a 3′ directed primer pair was used to study HCN1 (Genbank# AF247450/ NM_{—}053375) consisting of nucleotides 2391-2413 and 2589-2620. Primers used to amplify cyclophilin A (peptidylprolyl isomerase A) were: 157-182 and 496-521 of Genbank# NM_{—}017101. Products were cloned into pCR®4-TOPO vector (Invitrogen) and sequenced. Relative fluorescence was compared during the log-linear phase of amplification and copy number was calculated based on plasmid standard dilutions. Samples were normalized for differences in RNA extraction efficiency using simultaneously measured cyclophilin, by dividing measured cyclophilin values by the value for the largest amount of cyclophilin measured (recovered) per run, assumed to represent 100% extraction, thus converting cyclophilin values to a fraction of 1. Test samples were then divided by their respective cyclophilin fraction. Cyclophilin values did not vary significantly between control and SNL DRGs.

In-situ hybridization and Immunohistochemistry: Left (injured) and right (uninjured) L5 dorsal root ganglia were embedded in the same cryomold and processed simultaneously. A digoxigenin based detection system was used for in-situ hybridization (Braissant et al., (1998) Biochemica 1: 10-16). Labeled antisense and sense cRNA probes of HCN1, HCN2, HCN3 and HCN4 corresponded to bases 2391-2602, 1448-1880, 1907-2232 and 3459-3815 of sequences with GenBank accession numbers AF247450 (NM_{—}053375) (HCN1), AF247451 (HCN2), AF247452 (NM_{—}053685) (HCN3), and AF247453 (HCN4), respectively.

For immunohistochemistry, post-fixed sections were blocked in 5% normal goat serum then incubated with rabbit anti HCN antibodies overnight at 4° C. (anti-HCN1, 1:2000; Alomone Labs, 1:500; anti-HCN2, 1:500, Alomone Labs; anti-HCN3 1:1000). After secondary antibody application, sections were developed with a Vectastain Elite ABC kit (Vector Laboratories) and visualized with 3,3′-diaminobenzidine-tetrhydrochloride. Peptide or fusion protein pre-absorption and omission of primary antibodies were performed as negative controls.

Results: Quantitative real-time PCR comparison of mRNA levels for the four HCN subtypes in whole L5/6 DRGs revealed that, in sham operated DRGs, the rank order abundance of transcripts was HCN1>>HCN2>HCN3, HCN4. In the DRGs from nerve-ligated rats, we observed significant decreases in the amplicon in the 3′ end of the HCN1 molecule, but not the 5′ end of the HCN1 molecule. We also observed significant decreases in HCN2 mRNA, however HCN3/4 mRNA levels did not change (FIG. 9). In-situ hybridization using a 3′ directed probe sequence showed that the decreases in QPCR-detected HCN1 mRNA (3′ end) were reflected in decreases in visualized HCN1 message in neurons. Decreases in HCN2 mRNA were distinctly seen. The decreases in HCN1 and HCN2 message were not confined to any specific neuronal subpopulation, and the cellular distribution of HCN3 was unaltered.

Immunohistochemical staining of adjacent 10μ sections revealed that HCN1, 2 and 3 are co-localized in the membrane region of predominantly, but not exclusively, larger neuronal profiles. After nerve injury, changes in the distribution of immunoreactivity mirrored those seen in mRNA levels. An antibody directed toward the C-terminus of HCN1 revealed reduced membrane delineation in large neurons from nerve ligated rats in comparison to controls. An antibody directed toward the N-terminus also revealed reduced HCN1 immunoreactivity compared to controls. Marked decreases in HCN2 immunoreactivity were also apparent in injured DRGs compared to controls, in keeping with the PCR and in-situ data. While the distribution of HCN3 immunoreactivity suggested denser juxtamembranous staining in large neurons after injury, these changes were not clear enough to be considered definitive.

### EXAMPLE 10

### Abnormal Activity of HCN Pacemaker Channels in SNL rats vs. Sham Controls

Methods: Rats were prepared according to the above SNL model (L5 ligation). Sham rats were prepared identically, but without resection of the transverse process to avoid nerve trauma, and without nerve ligation. After 7 days, rats were killed by cervical dislocation and the ipsilateral L4 (no ligation) and L5 DRGs were quickly excised with fine forceps under a stereomicroscope and placed in ice cold Tyrode's containing pen/strep antibiotics.

Dissociation and culture of DRG neurons: DRGs from the L5 level of SNL, sham ligated and naive rats, and the L4 level of SNL rats were removed and maintained in ice-cold Tyrode's solution (140 mM NaCl, 4 mM KCl, 2 mM CaCl2, 1.3 mM MgCl2, 10 mM D-glucose, 10 mM HEPES, pH adjusted to 7.4 with NaOH) with additional 2 mM Ca⁺² (Tyrode's) prior to dissociation. Ganglia were transferred (1-2 per well) into 24-well tissue culture plates containing freshly prepared collagenase/protease solution (2 mg/ml collagenase (Sigma, type 1A) and 1 mg/ml protease (Sigma, type XIV) in Tyrode's containing pen/strep and gentamicin to dissociate the ganglia. After 45 min incubation in enzymes (37° C.; 5% CO2), ganglia were extensively washed 5 times at RT, for 5 min each wash, in 0.5 ml Tyrode's solution; separate Pasteur pipettes were used for each experimental condition. Ganglia were individually transferred into Eppendorf tubes containing 1 ml of DMEM (Gibco #11965-092) supplemented with 10% FBS (HyClone, #SH30070.03) and 1% pen/strep (Gibco 15070-063), and gently triturated to encourage dispersion of cells with fire-polished Pasteur pipettes of decreasing diameters. Cell suspensions (50-100 μl) were dropped on the center of freshly coated poly-D-lysine coverslips and incubated 30 min at 37 deg C. (5% CO2). Culture medium (0.5 ml) was then added to the wells. Just prior to plating, ˜50 μl sterile filtered poly-D lysine solution (300K, 1 mg/ml in water) was spread on the surface of 12 mm round #1 cover glass (VWR) and after 15 min at RT, was removed by extensive rinsing in water. Patch clamp recordings: The whole cell patch clamp technique(Hamill et al., (1981) Pflugers Arch 391: 85-100) was used to record voltage-activated currents from acutely dissociated DRG neurons with round or oval cell bodies without processes between 4 hours and 2 days after plating. Cells were visualized using a Nikon Diaphot 300 with DIC Nomarski optics. Cells were identified as small (16-31 μm), medium (32-42 μm) or large (>42 μm) (Villiere et al., (1996) J Neurophysiol 76: 1924-41) using a reticule in the eyepiece of the microscope. Only cells with diameters >42 μm were included in this study. The extracellular solution was Tyrode's. Recording electrodes were fabricated from borosilicate capillary tubing (R6; Garner Glass, Claremont, Calif.), the tips were coated with dental periphery wax (Miles Laboratories, South Bend, Ind.), and had a resistance of 2-2.5 MΩ when containing the following intracellular solution: 130 mM K-gluconate, 10 mM KCl, 3 mM MgCl₂, 10 mM hemi-Na-HEPES, 2 mM Mg-ATP, and 0.1 mM EGTA; pH 7.4, with dextrose added to achieve 290 mOsm as measured using a Wescor 5500 vapor-pressure (Wescor, Inc., Logan, Utah)). Tyrode's containing CsCl (3 mM) was bath applied to show inhibition of the hyperpolarization-activated current. ZD7288 was applied at 50 μM to determine the sensitivity of the current to this antagonist.

Current and voltage signals were detected and filtered at 2 kHz with an Axopatch 1D patch-clamp amplifier (Axon Instruments, Foster City, Calif.), digitally recorded with a DigiData 1200B laboratory interface (Axon Instruments), and PC compatible computer system and stored on magnetic disk for off-line analysis. Data acquisition and analysis were performed with PClamp software. Modulators of currents were applied by bath addition or from nearby puffer pipettes situated 2-3 cell diameters away. The puffer pipettes contained 0.05% fast green dye to indicate the extent of the plume upon pressure ejection of the contents.

Parameter determination: The same as described above in Example 3.

Results: Nearly all large neurons (>42 μm diameter) in control and SNL ganglia expressed currents consistent with Iₕ as demonstrated by activation by hyperpolarization in a voltage- and time-dependent manner, and efficacious block by extracellular Cs⁺ (3 mM) and ZD7288 (50 μM). The reversal potential of Iₕ currents was similar to previously reported values in SNL (−31.3±3.8, n=4) and control cells (−34.3±4.0, n=6). Large neurons from control DRG expressed Iₕ ranging from 0 to −21.3 pA/pF (normalized for cell capacitance). Most (˜58%) expressed less than 4 pA/pF (FIG. 10, hatched bars). A striking finding in SNL large L5 neurons was a shift in the Iₕ current density distribution such that only ˜8% expressed Iₕ<4 pA/pF (FIG. 10, solid bars). A population of neurons having low expression under control conditions appeared to have shifted to a high level of expression after insult. The threshold voltage for activation and the resting membrane potential were shifted to significantly more positive potentials in SNL neurons. There was a tendency for the SNL DRG neurons to have faster kinetics of activation when activated by voltage steps to less than −100 mV (FIG. 11). This difference is likely related to the shift in threshold for activation of Iₕ to more depolarized values in injured neurons.

### EXAMPLE 11

### Blockade of HCNs by Lidocaine

Lidocaine was tested for its effect on Iₕ expressed in dissociated L4 dorsal root ganglion neurons from uninjured rats to determine whether this well known Na channel blocker could have other mechanisms of action.

DRG were extirpated, dissociated and cultured 3-4 days on poly-D-lysine coated coverslips as described in Example 10. Iₕ was measured using the whole cell configuration of the patch clamp technique according to the methods described in Example 10 with the exception that the pipette solution was the solution used in Example 3. Neurons were challenged with a family of hyperpolarizing voltage pulses (−60 mV to −150 mV in increments of 10 mV) from a holding potential of −50 mV. Iₕ was determined at the end of 600 msec duration test pulses. Lidocaine was bath-applied at neutral pH and the percent inhibition of control Iₕ was determined after lidocaine achieved steady state block. Plotted is the steady state current observed at −134 mV as a percent of control after incubation of lidocaine at the indicated concentrations. Concentration-dependent block of Iₕ was seen with an ED50 of 23 micromolar. Lidocaine block was reversible. Data were obtained from 3 cells having control Iₕ densities of −1.5, −2.0 and −2.2 pA/pF.

### EXAMPLE 12

### Cloning and Purification of Recombinant Rat HCN C-terminus Polypeptides As Antigens

PCR primers were designed with BamHI site in the 5′ primer SEQ ID NO: 14, 5′ gcGGATCCccggacctcggggccgcccact 3′, and an EcoRI site in the 3′ primer SEQ ID NO: 15, 5′ gcGAATTCtcacatgttggcagaaatttgg 3′. PCR was run at 94° C. for 4 min, 40 cycles of 94° C. for 30 sec, 64° C. for 30 sec, 72° C. for 30 sec, and then 72° C. for 10 min with Chung DRG cDNA as template. Purified HCN3 fragment DNA resulting from PCR and pGEX-3X vector (Amersham) were double digested with BamHI and EcoRI. The digested HCN3 fragment was then fused in frame downstream of the GST gene on the digested pGEX-3X via DNA ligation. The obtained plasmid construct was transformed into E. coli DH5α competent cells (GIBCO), amplified in the transformed E. coli cells, and isolated from the cells. DNA sequence of the plasmid construct was verified by sequencing analysis. The correct plasmid construct was transformed into E. coli BL21 competent cells (Stratagene) for GST-HCN3 fusion protein expression. The fusion protein was subsequently purified from the BL21 transformants following standard GST-fusion protein purification protocols from the manufacture (Amersham). After further purification with dialysis, fusion protein was submitted to R&R Rabbitry (Stanwood, Wash.) for antibody generation.

The GST-HCN3 fusion protein comprises amino acids 712-780 of rat HCN3 (GenBank protein Id No: AAF62175), SEQ ID NO: 16, gprgrplsasqpslpqratgdgsprrkgsgserlppsgllakppgtvqpsrssvpepvtprgpq isanm.

Following a similar procedure, substantially purified GST-HCN1 fusion protein was also made and used for antibody development. The PCR primers used for cloning the HCN1 3′ fragment were: SEQ ID NO: 17, 5′ GCGGATCCCCACAGTCCACAGCACTGG 3′, and SEQ ID NO: 18, 5′ GCGAATTCTCATAAATTCGAAGCAAAACG 3′. The resulting GST-HCN1 fusion protein comprises amino acids 842-910 of rat HCN1 (GenBank protein Id No: AAF62173), SEQ ID NO: 19, tvhstglqagsrstvpqrvtlfrqmssgaippnrgvppappppaavqrespsvlnKdpdaekpr fasnl.

### EXAMPLE 13

### An Electrophysiological Assay Useful for Identifying Modulators of HCN Pacemaker Channel

The voltage clamp technique is used to identify blockers of HCN channel function. In one example, but not limited to this example, the whole cell configuration of the patch clamp technique is used to screen for compounds that block currents mediated by HCN channels expressed in mammalian cells, preferably a cell line that stably expresses HCN channels. In another example, oocytes expressing recombinant HCN channels are screened using the two electrode voltage clamp technique. The general methods are presented in Examples 2 and 3. The screen is performed by the following method: Current voltage relationships are determined under control conditions in which cells are challenged with voltage pulses from −40 to −150 mV. Subsequently, a repetitive single pulse protocol is applied to fully activate HCN channels by a voltage pulse to more negative than −110 mV. After the current amplitudes have stabilized, compound is bath applied at a concentration of 50 uM. Voltage pulses are applied continuously (e.g., every 10, 15, or 30 seconds) for 10 min since many compounds including ZD7288 have very slow onset of block. The amplitude of the steady state inward HCN-mediated current is determined and compared to the baseline amplitude. Current voltage relationship is determined after 10 min exposure to compound to identify subtle changes in voltage dependence.

### EXAMPLE 14

### A Binding Assay Useful for Identifying Modulators of HCN Pacemaker Channel

The binding of high affinity ligands can be useful for finding modulators of HCN pacemaker function. While there are currently no known modulators with submicromolar affinity, a number of selective compounds exist that may be useful for this purpose; such molecules include but are not limited to ZD7288, zatebradine, and antibodies specific to HCN proteins. Molecules or ligands known to interact with HCN proteins are radioactively labeled or conjugated to a fluorescent molecule for detection. These assays further require a source of HCN protein, such as HCN host cells (recombinant or native) exogenously expressing HCN protein or purified HCN protein from any of these sources, and negative controls that may include native tissue, isolated membranes from native tissue, etc.

An example of such an assay is given. Cells expressing HCN pacemaker, such as the cell line described in Example 2 are suspended in ice-cold external solution (in mM: 130 NaCl, 2 CaCl2, 4 MgCl2, 10 glucose, 20 HEPES, pH 7.3) with the inclusion of 0.1% BSA at 0.5×10⁶-2×10⁶ cells/ml. ¹²⁵I-ZD7288 (0.1-10 uM, TOCRIS), or other suitable labeled ligand, is then added to the cell suspension and the mixture incubated on ice for one hour with periodic gentle agitation. The mixture is centrifuged at 5,000×g for 5 minutes and the supernatant removed. Pellets are solubilized and radioactivity assessed in a gamma counter (Packard Bioscience). Specific ZD7288 binding is determined in the presence of 100 uM unlabeled ZD7288. Test compounds are included in the binding reaction and active compounds are those that enhance or inhibit radiolabelled ZD7288 binding to the HCN pacemaker protein expressing cells.

In the above example, the source of HCN pacemaker protein is an HCN pacemaker expressing cell line. It should be noted that this assay could also be performed with purified HCN pacemaker protein or microsomes containing HCN pacemaker proteins derived from native tissue or cell lines.

### EXAMPLE 15

### Cell-Based Fluorescence Assay for HCN Activity

A number of fluorescence assay formats can be utilized to measure HCN channel function. Since HCN channels are permeable to K⁺, Na⁺, and Rb⁺, fluorescence indicators or radioactive tracers for Na⁺ and K⁺, and non-radioactive AAS techniques or radioactive ⁸⁶Rb to determine Rb⁺ flux can be used to measure ion channel function in a cell-based system. Cells expressing HCN are grown in an optical bottom multi-well assay plate. The growth media is removed from the cells and the cells are loaded for 1 hour with a sodium sensitive fluorescent dye, e.g. SBFI (Molecular Probes). The dye solution is removed and the cells are placed in a small volume of sodium free solution. The assay plate is placed on a fluorescence plate reader and the cytoplasmic sodium concentration is measured. The extracellular sodium concentration is then raised to concentrations between 10 and 140 mM and the resulting increase in cytoplasmic sodium concentration is measured as a change in fluorescence. This assay takes advantage of the fact that HCN channels are permeable to sodium. Blockers of HCN channels such as Cs⁺, ZD7288 and zatebradine are included in some wells to determine the fraction of sodium influx through HCN channels compared to alternate sodium entry pathways. Test compounds are added to some wells and their effect on sodium influx is compared to control wells with no added compound and to wells containing known blockers of HCN. In another example, low concentrations of K⁺ can be added back to cells maintained in the absence of K⁺ for short incubation periods to inhibit influx of Na⁺ (see Pape, 1996). Upon addback of low concentrations of K⁺ (1-10 mM), Na⁺ permeability will be enhanced and Na⁺ influx can be measured. Mn⁺² in the low mM range may be used to shift the voltage of activation to the right and enhance the probability of opening (DiFrancesco et al., (1991) Experientia 47: 449-52). Alternatively, low pH can be used to shift the voltage dependence of activation such that channels will be open at more depolarized potentials (Stevens et al., (2001) Nature 413: 631-5.). In one example, HCN blockers can be identified by their ability to inhibit constitutive ion flux mediated by HCN channels under conditions where a fraction of channels are open at the resting membrane potential of the tested cell.

An alternate method to the one described above employs a fluorescent dye sensitive to membrane potential. Cells expressing HCN are grown in an optical bottom multi well assay plate. The growth media is removed from the cells and the cells are incubated with a membrane potential dye or the FRET pairs CC2-DMPE and DiSBAC₂(3) (Aurora Biosciences Corporation, catalog numbers 00 100 010 and 00 100 008, respectively). The assay can be conducted as above where the external sodium concentration is raised and the resulting sodium influx through HCN channels can be indirectly measured as a change in fluorescence of the membrane potential sensitive dye. Alternatively, HCN channels can be activated by hyperpolarizing cells by either altering the extracellular ionic composition or by adding a compound known to hyperpolarize cells. In this assay, membrane potential is monitored and the HCN component is identified by subtracting recordings made in the presence of an HCN blocker such as cesium or ZD7288. Alternatively, low pH can be used to shift the voltage dependence of activation such that channels are open at more depolarized potentials (Stevens et al., (2001) Nature 413: 631-5.). In one example, HCN blockers can be identified by their ability to decrease fluorescence (hyperpolarization) of the membrane potential.

What is claimed is:

1. A method for treating pain in a subject in need thereof, comprising administering to the subject a therapeutically effective dose of a composition that decreases the current mediated by an HCN pacemaker channel in a sensory cell of the subject.

2. The method of claim 1 wherein the composition is delivered in combination with an analgesic.

4. The method of claim 1 wherein said pain is an inflammatory pain.

5. The method of claim 1, wherein said composition decreases the expression of an HCN protein subunit in a sensory cell of the subject.

6. The method of claim 1, wherein said composition decreases open probability of an HCN pacemaker channel in a sensory cell of the subject.

7. The method of claim 1, wherein said composition decreases the conductance of an HCN pacemaker channel to ions in a sensory cell of the subject.

8. The method of claim 1, wherein said composition is an inhibitor of an HCN1 or HCN3 channel.

9. The method of claim 1 wherein the pain is selected from the group consisting of carpal tunnel syndrome pain, back pain, neck pain, sciatica, intercostal neuralgia, opioid resistant pain, headache, cluster headache, migraine, trigeminal neuralgia, arthritis, osteoarthritis, and cancer-related pain.

10. The method of claim 1, wherein said composition is selected from ZD7288, ZM-227189, Zatebradine, DK-AH268, alinidine, and ivabradine.

11. The method of claim 1 wherein said other analgesic is selected from morphine or other opiate receptor agonists; nalbuphine or other mixed opioid agonist/antagonists; tramadol; baclofen; clonidine or other alpha-2 adrenoreceptor agonists; amitriptyline or other tricyclic antidepressants; gabapentin or pregabalin, carbamazepine, phenytoin, lamotrigine, or other anticonvulsants; and/or lidocaine, tocainide, or other local anesthetics/antiarrhythmics.

12. A method for treating pain in a subject in need thereof, comprising administering to the subject a therapeutically effective dose of a composition that decreases the expression of an HCN subunit in a sensory cell of the subject.

13. The method of claim 12 wherein the composition comprises an antisense nucleic acid or siRNA molecule specific for an HCN gene and wherein the antisense nucleic acid or siRNA molecule specifically suppresses HCN gene expression.

14. The method of claim 13 wherein said antisense nucleic acid or siRNA molecule specifically suppresses HCN1 or HCN3 gene expression.

15. An antibody that selectively binds to the carboxy-terminus of a HCN protein.

16. The antibody of claim 15 wherein the antibody selectively binds to the carboxy-terminus of a HCN1 or HCN3 protein.

17. A method of identifying a compound useful for treating pain, comprising the steps of:
(a) contacting a test compound with an HCN pacemaker protein; and
(b) determining the ability of the to decrease a current mediated by an HCN pacemaker channel.

18. The method of claim 17 further comprising the step of testing the compound in at least one animal model for pain.

19. The method of claim 17, wherein the protein is an HCN1 or HCN3 pacemaker subunit.

20. The method of claim 17 wherein said HCN pacemaker protein is substantially purified.

21. The method of claim 17 wherein said HCN pacemaker protein is associated with a membrane.

22. The method of claim 17 wherein said HCN pacemaker protein is expressed from a host cell.

23. A method of identifying a compound useful for treating pain, comprising the steps of:
(a) contacting a test compound with a regulatory sequence for an HCN pacemaker gene or a cellular component that binds to the regulatory sequence for an HCN pacemaker gene; and
(b) determining whether the test compound decreases the expression of a gene controlled by said regulatory sequence.

24. The method of claim 23 further comprising the step of testing the compound to at least one animal model for pain.

25. The method of claim 23 wherein the regulatory sequence is a regulatory sequence for an HCN1 or HCN3 gene.

26. The method of claim 23 wherein the gene controlled by the HCN regulatory sequence is a reporter gene.

27. The method of claim 23 wherein the gene controlled by the HCN regulatory sequence is an HCN gene.

28. The method of claim 23 wherein the regulatory sequence and the controlled gene thereof are inside a host cell.

29. A method of identifying a compound useful for treating pain, comprising the steps of:
(a) combining a test compound, a measurably labeled ligand for an HCN pacemaker protein, and an HCN pacemaker protein; and
(b) measuring binding of the compound to the HCN pacemaker protein by a reduction in the amount of labeled ligand binding to the HCN pacemaker protein.

30. The method of claim 29 additionally comprising the step of testing the compound in at least one animal model for pain.

31. The method of claim 29 wherein said HCN pacemaker protein is substantially purified.

32. The method of claim 29 wherein said HCN pacemaker protein is associated with a membrane.

33. The method of claim 29 wherein said HCN pacemaker protein is expressed in a host cell.

34. The method of claim 29 wherein said protein is an HCN1 or HCN3 pacemaker protein.

35. A method for treating pain in an animal comprising the step of administering to the animal a composition including one or more inhibitors of an HCN pacemaker protein.

36. A method for treating inflammatory pain comprising administering to an animal a composition comprising one or more inhibitors of an HCN pacemaker protein.
